(19) United States
(12) Patent Application Publication
(10) Pub. No.: US 2016/0270878 A1
(43) Pub. Date: Sep. 22, 2016

(54) DENTAL DEVICE AND SUCTION DEVICE

(71) Applicant: Awestruck Dental, LLC, Telluride, CO (US)

(72) Inventor: Richard Eustis Fulton III, Grand Junction, CO (US)

(21) Appl. No.: 15/084,230

(22) Filed: Mar. 29, 2016

Related U.S. Application Data
(63) Continuation-in-part of application No. 14/504,518, filed on Oct. 2, 2014.
(60) Provisional application No. 61/961,106, filed on Oct. 2, 2013.
(60) Provisional application No. 61/967,319, filed on Mar. 13, 2014.
(60) Provisional application No. 61/997,780, filed on Jun. 9, 2014.
(60) Provisional application No. 62/178,129, filed on Mar. 30, 2015.
(60) Provisional application No. 62/274,625, filed on Jan. 4, 2016.

(51) Int. Cl. A61C 5/00; A61C 5/14; A61M 1/00; A61C 17/06

(52) U.S. Cl. CPC A61C 5/007; A61C 17/043; A61C 5/14; A61M 1/008

(57) ABSTRACT

A device for helping maintain a mouth of a patient in an open position during a dental procedure may include a novel suction device that preferentially aspirates fluids and not the tissues of the mouth.

### CROSS-REFERENCE TO RELATED APPLICATIONS

The present application is a continuation-in-part of co-pending U.S. patent application Ser. No. 14/504,518, filed on Oct. 2, 2014 [attorney docket no. 47145-703.201], which claims priority to U.S. Provisional Patent Application Ser. Nos. 61/961,106, filed on Oct. 2, 2013 [attorney docket no. 47145-703.101], 61/967,319, filed on Mar. 13, 2014 [attorney docket no. 47145-703.102], and 61/997,780, filed on Jun. 9, 2014 [attorney docket no. 47145-703.103]; and, this application also claims the benefit of U.S. Provisional Application Ser. Nos. 62/178,129, filed on Mar. 30, 2015 [attorney docket no. 47145-703.104], and 62/274,625, filed on Jan. 4, 2016 [attorney docket no. 47145-703.105], the entire disclosures of which are incorporated herein by reference.

The present application is related to co-pending U.S. patent application Ser. No. 14/504,518, filed on Oct. 2, 2014, entitled “Patient Controlled Dental Device and Method” [attorney docket no. 47145-703.201], and PCT Application No. PCT/US2014/058904, filed on Oct. 2, 2014, entitled “Patient Controlled Dental Device and Method” [attorney docket no. 47145-703.601], the entire disclosures of which are incorporated herein by reference.

### BACKGROUND

Dental procedures are very common and are necessary for proper dental health, whether it be dental fillings, dental crown applications, root canals, orthodontic work, periodontal surgery, dental cleaning, and many other procedures. One common problem with these dental procedures is that the patient must maintain an open mouth for extended periods of time. The dentist, dental assistant, or dental hygienist must have adequate exposure to access the site to be treated. While opening of the mouth for short periods of time is not problematic for most patients, many, if not most, patients do have difficulty maintaining an open mouth for the extended periods of time required for many dental procedures. All dental patients have at least some difficulty opening their mouths for prolonged periods, as this prolonged opening fatigues the muscles and becomes uncomfortable. Most patients just accept that going to the dentist is uncomfortable and just tolerate the discomfort. Fifty percent of patients have significant difficulty keeping the mouth open for any procedure. This percentage increases with the length of time of the procedure. Elderly, young, and patients on psychotropic drugs have more difficulty that others, but virtually all patients have discomfort opening the mouth for any period of time.

This rather high figure may reflect the prevalence of temporomandibular joint disorders (TMD) in the general population. Epidemiological studies have revealed an average of 30%-44% prevalence of TMD in the general population. All of these patients will experience difficulty at the dentist, with either limited mouth opening or with pain and discomfort in the jaw muscles from straining to keep the mouth open or both. There is also a subset of the population that does not have TMD but that does have difficulty and/or pain with limited mouth opening, due to other causes of muscle tightness. Examples of such causes of difficulty with mouth opening include bruxism, clenching, stress, psychological issues, and others. This segment accounts for as much as 20% of the population. Hence, well over 50% of the population may have difficulty with prolonged mouth opening for dental procedures.

The typical reason for difficulty with prolonged mouth opening is muscle fatigue and spasm that initially becomes uncomfortable but progresses to become quite painful. The human mouth is a simple hinge, with the mandible articulated with the skull at the temporomandibular joint. The muscle systems that cause the mandible to close are mainly the large masseter muscles and the temporalis muscles. These are robust and responsible for forceful chewing, mastication, clenching, bruxing, etc. The corresponding muscles responsible for opening the mouth are mainly the small and delicate pterygoid muscles. This causes a disparity in the opposing muscle groups, which is not of importance in the normal function of the mouth during usual activities such as eating, drinking, speaking, and the like. However, prolonged opening of the mouth is affected by, and very difficult because of, the size, disparity, and arrangement of the opposing muscle sets. The smaller pterygoid muscles tire after several minutes of prolonged opening, causing discomfort and actual pain.

This creates a dilemma, as the dental practitioner must have access to the oral cavity to perform the necessary task, but frequently the simple act of holding the mouth open is uncomfortable to the patient. Devices that assist in holding the mouth open have been in use for many years for this reason. These consist of bite blocks, which are simple wedge devices placed between the teeth to prop open the mouth by creating a consistent space between the upper teeth and lower teeth by depressing the mandible. Bite blocks are used on one side of the jaw or the other to allow access for procedures, and this asymmetrical use can strain the temporomandibular joints, often triggering joint pain. Use of a bite block also creates a persistent and continuous stretching of all the muscles that affect the depression of the mandible, i.e., the pterygoids, or elevation of the mandible, i.e., the masseters and the temporalis groups. These devices offer no period of relaxation for the muscles to recover from the stress of the continuous stretching. This lack of a relaxation period can result in pain and discomfort to the patient. The bite blocks also occupy space within the oral cavity and impede access or exposure to the specific area that needs attention or treatment.

Other known devices have been developed that prop the mouth open but are almost universally awkward to employ, as they also frequently inhibit access to all parts of the oral cavity and the dental practitioner must navigate around these devices to perform the necessary and intended maneuvers. They also provide a continuous and persistent stretching of the musculature, which results in discomfort while attempting to assist in keeping the mouth open. The bite blocks commonly used actually stimulate contraction of the muscles responsible for closing the mouth and accentuate the problems and difficulties.

In U.S. Pat. No. 6,030,217, Fletcher describes a device comprised of a flexible mouth piece that fits over the lower front teeth and an elongated flexible member attached at one end to the mouth piece and the other end to a handhold object. Fletcher's device may suffer from limitations that preclude optimal functioning to assist with opening the mouth and maintaining it open in a comfortable position in a safe manner. For instance, the mouth piece has sufficient flexibility to permit the mouth piece to bend and release from the lower teeth when the flexible member is subjected to a selected threshold tension. This, in essence, means that the mouth piece will typically flex and become disengaged from the lower teeth if the patient exerts more downward force than the mouth piece will tolerate. With significant downward force being exerted and an abrupt release by the mouth piece, the mouth piece may be forcefully be propelled or pulled downward toward the hand. As soon as the trailing edge clears the teeth, it may engage the lower lip with significant abrupt downward force, and at the very least, bruise the lower lip if not lacerate it. As well, the mouthpiece may be propelled as a missile toward the hand, where it may impact the hand, fingers, or other body part or even the dentist or dental assistant. Just the thought of a piece of plastic flying about the dental operatory, contaminating equipment and personnel with saliva, enough to discourage the use of such a device.

Additionally, since any incremental tension may be at least partially, if not mostly, absorbed by the flexible mouth piece of Fletcher, rather than transferred to the mandible, the mouth piece will bend or deform, instead of providing additional consistent, graded and gradual downward pressure on the mandible. Slow and gradual increases in tension downward are often needed for maximum relaxation and stretching. Hence, the flexible mouthpiece of Fletcher may be limited for this reason as well.

Moreover, the elongated flexible member of Fletcher can create an unlimited number of angles and directions in which the downward force may be directed. The elongated flexible member of Fletcher may introduce variability in the use of the device, which also can be a safety issue. The teeth may be accustomed to downward pressure and are very stable when pressure is in the same plane as the long axis of the tooth. If the force is downward and forward, or downward and to one side or the other, the tooth or teeth may be loosened or tilted. Marginally loose teeth from periodontal disease or other causes could be loosened even more than at their resting state. The temporomandibular joint (TMJ) may also be damaged by inappropriately directed forces. Tension forward or outward may even dislocate the TMJ, and tension to the side may cause TMJ dysfunction or worsen TMJ preexisting conditions. With so many variations in the direction of the force, it may be exceedingly difficult to align the flexible member, the mouth piece and the handhold object so that the proper direction of the force is maintained during the entire dental procedure, which may last for an hour or longer. Hence, the flexible member of Fletcher may be convenient to connect the mouth piece with the handhold object, but it may be dangerous to the patient and the staff for a number of reasons, and it may create unintended problems and difficulties.

Additionally, there may be a need for devices or methods that increase the degree of opening of the mouth beyond passive opening using the muscles of the jaw or opening with a simple assist device. Not only do patients experience discomfort from having the jaw open for protracted periods, but the limited exposure to the dental operative field constantly constrains the dental practitioner. The ability to open the mouth maximally diminishes with the time the mouth is open, as the muscles fatigue and less than a fully open position is sought for comfort reasons. The practitioner is forced to operate and perform procedures within the mouth, which is basically a small hole, and that hole becomes smaller with time. The wider the patient is able open the teeth, the better the exposure for the dental practitioner and the less tedious the work becomes.

No known prior art devices are targeted improving the degree of opening the mouth or providing more exposure as the procedure progresses. Bite blocks act as props, and the Fletcher device may either prop or distract the mouth open, but this is where these devices stop. The bite blocks are forced between the upper and lower molars and may actually trigger a reflex to bite down. They certainly do not continue to improve the degree of opening. The Fletcher device may assist in opening the mouth and may reduce the discomfort despite the significant limitations and safety issues it presents, but it does not continue to improve the opening of the mouth and continue to improve the exposure available to the dentist. While the prior art includes devices that attempt to address the mouth-opening problem, all of these devices have shortcomings, as discussed herein.

Another major limitation of known prior art devices is that they are all placed into the mouth, in addition to other devices used in a procedure. In other words, they are additive devices and occupy valuable space in the mouth, which limits the access by the dentist or hygienist in addition to the other tools that may be used. For example, while many different tools and devices may be used during a dental procedure, suction is used almost universally in dental procedures. Hence, the known prior art devices must be inserted in addition to the suction apparatus. This may become awkward and confusing, for example, if the patient is responsible for the suction with one hand and the Fletcher prior art device with the other hand. More importantly, the space occupied by a combination of suction tube and the prior art devices may well impede access to the mouth by the dentist. There is the probability of simply having too many items in the mouth at one time to allow easy access.

Therefore, it would be very desirable to have improved devices and methods for maintaining a mouth in an open position for prolonged periods of time for dental procedures. Ideally, these devices and methods would provide improvements over the prior art devices and methods and would reduce or eliminate at least some of the discomfort and pain of maintaining an open jaw. Also ideally, such devices and methods should work well for patients while not overly obstructing the dentist's access to the mouth for performing procedures. The embodiments described herein will meet at least some of these objectives.

### SUMMARY

To solve the dilemmas described above, of the need to maintain an open mouth for an extended period of time, to open the mouth beyond that which is achievable from natural active opening, and to do both comfortably, the devices and methods described herein may rely on several basic physiologic principles. By exploiting these principles, the dental procedure can be more comfortable for the patient and can be performed by the practitioner more quickly and without significant interruptions. The degree that the mouth can open comfortably will also be enhanced, providing better access to the mouth for the dental practitioner.

One of the physiologic principles of which the present devices and methods may take advantage is that the distraction of the mandible may be intermittent, rather than the consistent and persistent distraction necessary with the prior art devices. This intermittent distraction may allow the musculature to rest occasionally and temporarily recover from the stress of the stretching. A brief change in the degree of stretching is often all that is frequently needed to prevent the discomfort.

Another physiologic principle is that downward pressure on the mandible actually relaxes the large masseter, temporalis, and internal pterygoid muscles that close the mouth via a reflex. This is the same reflex that relaxes these muscles when chewing exerts downward pressure on the mandible and stops the chewing motion when the teeth touch.

Hence, distraction of the mandible downward will not only aid the opening muscles of mastication (suprahyoid and lateral pterygoid muscles), but can obviate much of the contraction of the much larger and more powerful closing muscles of mastication by stretching and relaxing them.

Another principle is that continued low level, graded, and incremental traction on a muscle will relax and stretch the muscle, thus elongating the muscle and inhibiting reflex contraction of the muscle. Continued low level traction or stretching will continue to elongate the muscle. Hence, a technique and device that employs at least some of these physiologic principles may cause the mouth to open wider than possible with just active opening (without an assist) by the patient.

Another principle is that the maximum distraction of the mandible will occur when the patient temporarily relaxes the distractive force when the dental practitioner is not directly engaged in intra oral manipulations or functions. This may happen intermittently, when the practitioner must change implements or instruments, drill bits, scalers, curettes, probes, etc. During this brief interlude, the patient can temporarily relax the distraction to provide relief to the stretched muscles. Subsequent stretching will be even more effective as a result of this intermittent relaxation, which cannot occur with the use of a bite block. It may also be problematic to allow relaxation using the Fletcher device, because the mouth piece may become dislodged if all of the tension on the flexible member is relaxed, as there would be no force to hold the mouth piece adjacent to the teeth. The inability to accommodate these important maneuvers often prevent the prior art devices from functioning optimally.

Moreover, embodiments of the present disclosure may allow the dental practitioner to encourage or prompt the patient to provide more distraction at certain times. Hence, while the patient may control the force and degree of depression of the mandible, the dental practitioner has input, so adequate exposure is provided. This feedback loop may be essential in providing adequate exposure to the practitioner while providing patient comfort. The feedback loop may be another physiological principle not shown to be exploited in the prior art devices.

The feedback loop may also go from patient to practitioner. The patient frequently is unable to articulate any meaningful message to the practitioner if prior art devices are used, essentially using grunts and garbled words. The devices of the present disclosure would allow verbal and other communication, as will be described further below.

Aspects of the present disclosure provide a device for helping maintain a mouth of a patient in an open position during a dental procedure. The device may include a tooth engaging portion comprising at least one groove for accepting at least one lower tooth of the patient's lower jaw, an extension portion that extends away from the tooth engaging portion, and handle that extends down from the extension portion and ends in a caudal end. The caudal end of the handle and the groove of the tooth engaging portion may lie along a longitudinal axis drawn through a center of the groove. The handle and the extension portion may comprise a solid, one-piece construct. At least part of the tooth engaging portion may comprise the solid, one-piece construct. The tooth engaging portion may comprise a distal end of the solid, one-piece construct, and a piece of material attached to the distal end of the one-piece construct, wherein the piece of material is softer than the one-piece construct.

The extension portion may be curved. The extension portion and the tooth engaging portion may be forked, and a space between two prongs of the tooth engaging portion and the extension portion may be configured to accept a suction tube. The caudal end of the handle may comprise a finger loop through which a finger of the patient may be extended to facilitate application of downward force. A traction member may further be included to be removably coupled with the handle, for applying downward force to the handle. For example, the traction member may be a weight. The device may also additionally include a suction member coupled with the tooth engaging portion and/or the extension portion. Another feature may be a tissue displacement member coupled with the tooth engaging portion and/or the extension portion, for displacing the cheek and/or tongue of the patient. Yet another feature may be a tensiometer coupled with the device to measure downward force applied to the device.

Aspects of the present disclosure may also provide a method of maintaining a mouth of a patient in an open position during a dental procedure. These methods may involve placing a groove of a tooth engaging portion of a mouth distraction device over at least one lower tooth in a lower jaw of the patient's mouth, and pulling down on a handle of the mouth distraction device in a direction along a longitudinal axis of the lower tooth to maintain the lower jaw in an open position relative to an upper jaw of the patient's mouth. The groove of the tooth engaging portion of the mouth distraction device and the handle of the tooth distraction device may lie along a common, distraction device longitudinal axis, and the distraction device longitudinal axis may lie along the longitudinal axis of the at least one tooth during the pulling step.

The pulling step and/or the placing step may be performed by the patient. After the pulling step, the pulling force from the handle may be released to allow the lower jaw to relax and the mouth to at least partially close, and the pulling step may be repeated to reopen the mouth. The groove may be placed over multiple lower front teeth.

The methods may also involve applying suction in the patient's mouth, using a suction device incorporated into or attached to the mouth distraction device. Also, a weight may be attached to the handle of the mouth distraction device to apply downward force. The cheek and/or tongue of the patient may also be displaced using a tissue displacement member incorporated into or attached to the mouth displacement device. A lip retractor means may be attached to or incorporated into the mouth distraction device to displace the lips laterally, vertically, or other directions to provide more exposure to the oral cavity. A tensiometer coupled with the device may be used to measure downward force applied to the device. After the pulling step, a suction device may be passed through an opening in the mouth distraction device and into the patient's mouth, and suction may be applied in the mouth with the suction device, while the distraction device maintains the mouth in an open position.

The mouth opening devices described herein may be constructed for children in more or less the same configurations as described herein, but smaller to accommodate the smaller mouth and teeth of children. Moreover, attachments may be provided to the device to create a more visually appealing device to children.

These and other embodiments and aspects are described in greater detail below, in relation to the attached drawing figures.

### BRIEF DESCRIPTION OF DRAWINGS

FIGS. 1A and 1B are side and top views, respectively, of a mouth opening device, according to many embodiments;

FIGS. 1C and 1D are side/cross-sectional and perspective views, respectfully, of a mouth, illustrating operation of the mouth opening device of FIGS. 1A and 1B, according to many embodiments;

FIG. 2 is a sagittal view of a mouth and a side view of a mouth opening device in position in the mouth, according to many embodiments;

FIGS. 3A, 3B, and 3C are side, front and bottom views, respectively, of a mouth opening device, according to many embodiments;

FIG. 3D is a close-up, bottom view of a tooth piece of the device of FIGS. 3A-3C;

FIG. 4 is a posterior perspective view of a mouth opening device, according to many embodiments;

FIG. 5 is a posterior perspective view of a mouth opening device, according to many embodiments;

FIGS. 6A, 6B, and 6C are side, close-up/front and close-up/side views, respectively, of a mouth opening device, according to many embodiments;

FIGS. 7A, 7B, and 7C are side, front and posterior perspective views, respectively, of a mouth opening device with components for connecting with a suction device, according to many embodiments;

FIGS. 8A, 8B, and 8C are side, front and posterior perspective views, respectively, of a mouth opening device with components for connecting with a suction device, according to many embodiments; and

FIGS. 9A, 9B, 9C, 9D, and 9E are side, front, close-up/perspective, end-on/cross-sectional, and posterior perspective views, respectively, of a mouth opening device with a built-in suction channel for connecting directly with a suction hose, according to many embodiments.

FIG. 10 is a top down perspective of the lower mouth with a mouth opening device, according to many embodiments.

FIG. 11 is a top down perspective of the lower mouth with a mouth opening device, according to many embodiments.

FIG. 12 is a top down perspective of the lower mouth with a mouth opening device, according to many embodiments.

FIGS. 13, 14, and 15 are side perspectives of mouth opening devices, according to many embodiments.

FIG. 16 is an oblique perspective of the device of FIG. 11.

FIG. 17A is a side perspective view of a mouth opening device, according to many embodiments.

FIG. 17B is a top down perspective view of the lower mouth with the mouth opening device of FIG. 17A placed therein.

FIG. 18 is a magnified perspective view of the tooth capturing end and suction tube of a mouth opening device, according to many embodiments.

FIGS. 19A and 19B show cross-sectional views of a suction tube apparatus for use with a mouth opening device, according to many embodiments.

FIGS. 20A, 20B, and 20C show sectional views of various ways to anchor a tubular braid with a suction tube apparatus for use with a mouth opening device, according to many embodiments.

FIG. 21 shows a top down perspective view of the lower mouth with a mouth opening device, according to many embodiments.

FIG. 22 shows a side perspective view of a mouth opening device with a suction apparatus coupled thereto at two suction ports, according to many embodiments.

FIG. 23 shows a side view of a main body, including its handle, of a mouth opening device, according to many embodiments.

FIGS. 24A and 24B show side views of the tooth hook of the mouth opening device of FIG. 23.

FIGS. 25A and 25B show side views of the handles of various mouth opening device, according to many embodiments.

FIGS. 26A, 26B, and 26C show side views of the handles of various mouth opening device, according to many embodiments.

FIGS. 27A, 27B, and 27C show end views of the handles of various mouth opening device, according to many embodiments.

FIGS. 28A, 28B, and 28C show end views of the handles of various mouth opening device, according to many embodiments.

FIGS. 29A, 29B, and 29C show end views of the handles of various mouth opening device, according to many embodiments.

FIGS. 30A and 30B show end views of a tooth hook of a mouth opening device that can swing, according to many embodiments.

FIGS. 31A, 31B, and 31C show side perspective views of a mouth opening device with a secondary tether that can be pulled downward, according to many embodiments.

FIGS. 32 show side perspective views of a mouth opening device with an elastic zig zag element that can be pulled downward, according to many embodiments.

FIG. 33 shows a side perspective view of a handle of a mouth opening device with a “V” grip, according to many embodiments.

FIGS. 34A and 34B show magnified perspective views of a tooth hook end of a mouth opening device with a swivel mechanism for its suction port, according to many embodiments.

FIG. 35 shows a side perspective view of a mouth opening device with a mist collector, according to many embodiments.

FIG. 36 shows a side perspective view of an ultrasonic scaler hand piece with a mist collector, according to many embodiments.

FIG. 37A is a transverse cross-section view of an exemplary embodiment of a suction tube for insertion into the mouth.

FIG. 37B is a transverse cross-section view of an exemplary embodiment of a suction tube for insertion into the mouth.

FIG. 38 is a transverse cross-section view of an exemplary embodiment of a multi-lumen suction tube.

FIG. 39A is a transverse cross-section view of another exemplary embodiment of a suction tube.

FIG. 39B is a transverse cross-section view of another exemplary embodiment of a suction tube.

FIG. 40A shows an exemplary embodiment of a helical ribbon of a suction tube.

FIG. 40B shows an exemplary embodiment of suction tube comprising a helical ribbon.

FIG. 41 is a schematic illustration of an exemplary embodiment of a suction tube.

### DETAILED DESCRIPTION

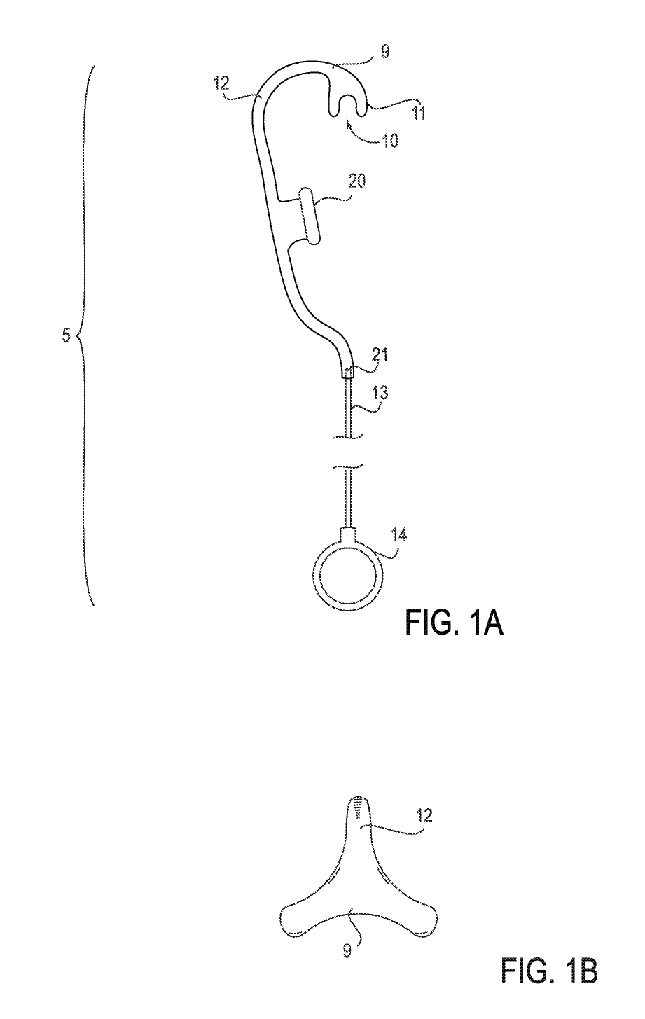

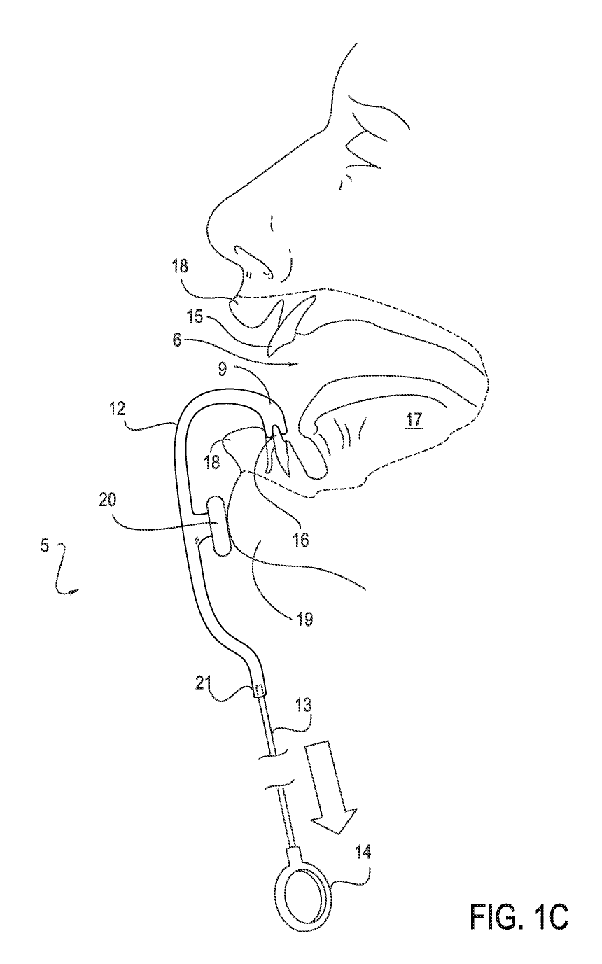

FIGS. 1A-1D illustrate a mouth opening device 5, from various perspectives (side view, top view, side view in a cross-sectional view of a mouth, and perspective view in a mouth, respectively). As illustrated in FIG. 1A, the device 5 may include a tooth piece (or “tooth component”) 9, which may fit over at least one lower tooth, an extension component 12, which may extend out of the mouth and over the lower lip, extending caudally to a point under the chin and in more or less the same coronal plane as the two lower front teeth, and a flexible component (or “flexible member”) 13, which may extend from at attachment point 21 at the bottom (or “caudal”) end of the extension component 12 further caudally and may allow a patient to provide traction on the device 5. The flexible component 13 may be attached at its caudal end to a handle or ring 14, which the patient may grip during use. The tooth piece 9 may fit over one or more lower teeth and includes a channel 10 with lips 11 on each side of the channel 10. The extension component 12 may include a chin pad 20 and may have a wide variety of lengths, shapes and sizes. For example, the extension component may have an overall length of between about 2 inches and about 24 inches.

FIG. 1B is a top view of the device 5 and demonstrates the tooth component 9, which fits over the teeth, and the upper part of the extension element 12.

FIG. 1C is a midline cross-sectional view of a mouth 6, showing an upper tooth 15, a lower tooth 16, the tongue 17, the lips 18 and the chin 19. FIG. 1C illustrates how the tooth component 9 may rest over the lower tooth 16, with the chin pad 20 resting against the chin 19. Pulling the handle/ring 14 downward may cause the mouth to open and the chin 19 to lower relative to the skull and upper mouth. FIG. 1D is a perspective view of the mouth 6 and device 5 from FIGS. 1A-1C.

As illustrated in FIGS. 1C and 1D, the device 5 may be used for deflection of the jaw or chin 19. The chin pad 20 may be included or not included. Pulling downward on the jaw can stimulate a reflex that relaxes the closing muscles of the jaw, so the action of the device can not only aid the opening muscles, but can also relax the opposing closing muscles. By combining the assistance to the small opening muscles with traction and relaxing the large opposing closing muscles at the same time, the patient may be able to comfortably maintain an open mouth for a protracted period of time.

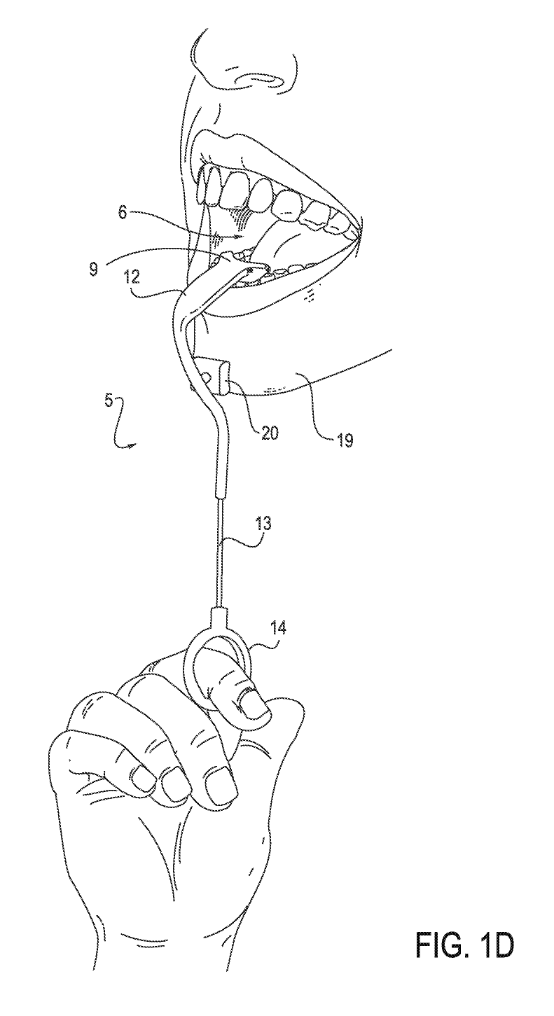

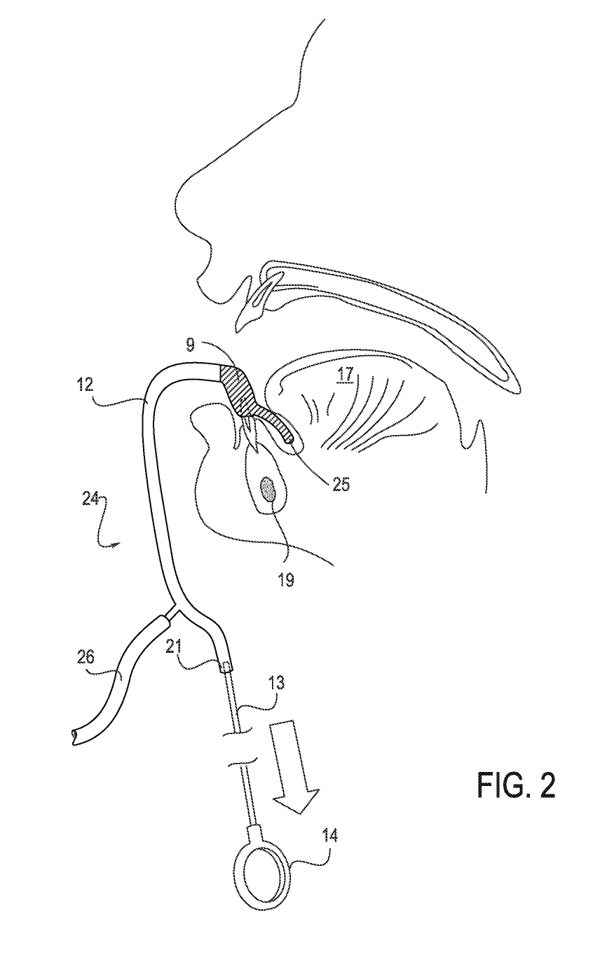

It may be important that the direction of the traction be in the longitudinal axis of the teeth, as they may be subjected to this same force daily during chewing and other activities. They may be accustomed to this direction of force. Traction anteriorly or obliquely forward may cause a tilt of the tooth and may loosen them. Any force directed other than along the long axis of the engaged teeth may be expected to displace the teeth and loosen them, especially if that force was exerted for 30 minutes or longer. The devices described herein may be designed so that the direction of force is along the vertical axis of the teeth, hence avoiding this potential problem. Additionally, traction outward or from posterior to anterior may create forces detrimental to the temporomandibular joint (TMJ). The device 5 may be designed so that the direction of force is along the vertical axis of the teeth, hence avoiding this potential problem. This direction of force is best illustrated in FIG. 1C and in FIG. 2 described below.

Referring now to FIG. 2, a sagittal view of the mouth is shown, along with a mouth opening device 24. The attaching component 9, extension element 12, flexible component 13 and ring 14 of the mouth opening device 24 may be the same or similar to the corresponding components of the mouth opening device 5 and other mouth opening devices described herein. A difference may be that the extension element 12 of the mouth opening device 24 does not include a chin pad. The device 24 may also include a suction channel within the extension element 13 and attaching component 9. A suction extension 25 may extend from the attaching component 9 to a point under the tongue 17 where most of the saliva exits the salivary ducts. There may also be a suction tubing 26, attached to the extension member 12 on one end and available suction machine on the other end. This combination of comfortable distraction of the jaw 19 and suction of saliva can be appealing for several reasons, one of which is that it may obviate the need for recurrent interruptions of the procedure to aspirate saliva from the mouth. It may even obviate the need for a dental assistant.

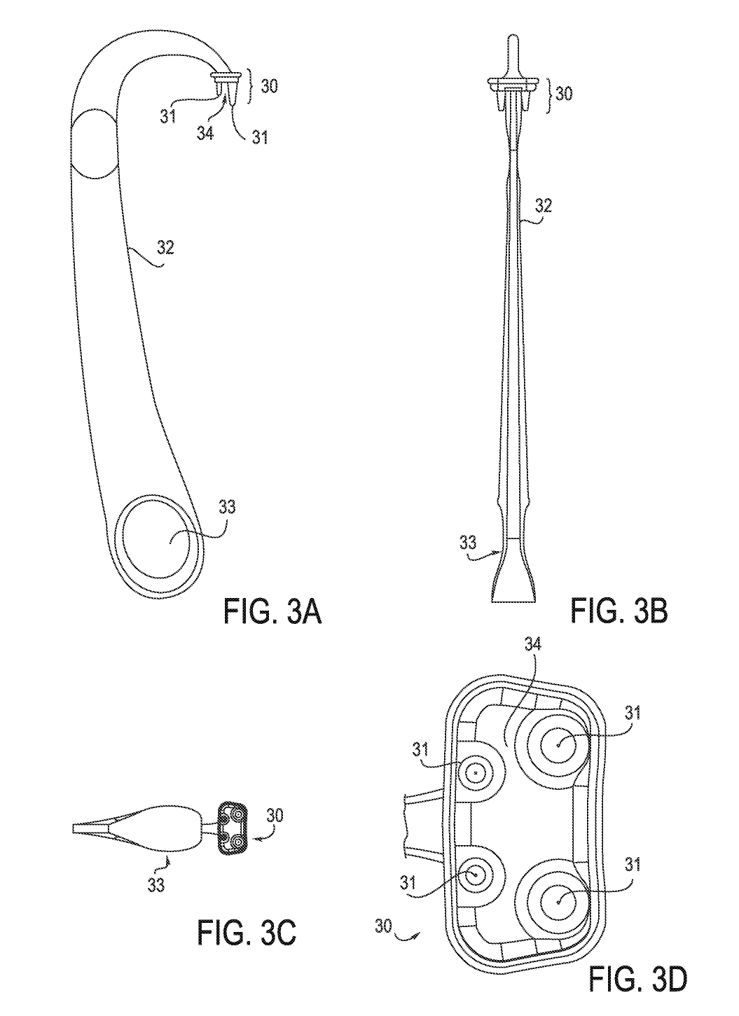

With reference now to FIGS. 3A-3D, a mouth opening device 28 is shown, which may not include the flexible member of the mouth opening devices 5 and 24 described herein. Otherwise, the components of the mouth opening device 28 may be the same or similar in many respects to that of the other mouth opening devices described herein and vice versa. The device 28 may include a tooth piece component 30, which may fit over at least one lower tooth, an inflexible extension component 32 (or “handle”), which may extend out of the mouth and over the lower lip extending caudally to a point under the chin to a point approximately in the same coronal plane as the two lower front teeth, and a gripping member 33 (or “finger loop”) for gripping and applying downward traction by the patient. The gripping member 33 may be a finger hole, but any other types, sizes and configurations of gripping portions may also be used. The tooth piece 30 may be made of a softer durometer material, formed into small posts 31, which may form the small groove 34 to accommodate the teeth and provide stability. The posts 31 may be arranged so that the teeth may fit between them, either side to side or front to back, so that the device 28 may be used either in the midline of the patient, i.e., directly over then under the chin, or from the side of the mouth, i.e., ninety degrees from the midline position. This latter position may place the curve of the handle 32 out of the way of the dental practitioner. Moreover, the posts 31 may be supplanted or augmented by deeper grooves (not shown) to receive the teeth. These grooves may be oriented transversely within the tooth piece 30 so that the device 28 may be positioned directly in the midline and in an anterior posterior or sagittal direction within the tooth piece 30, so that the device 28 may be positioned over the front teeth ninety degrees from midline. The device 28 may comprise either of the transverse or sagittal grooves or both and may be used from directly in the midline to the side of the mouth.

The handle 32 is typically relatively solid and inflexible. The handle 32 can add stability to the device 28 and may help ensure that the direction of the force applied by the patient is in the long axis of the tooth. The finger loop 33 of the handle 32 may be directly beneath the tooth, whether the handle 32 and device 28 are positioned in the midline or ninety degrees from midline, so the direction of force applied by the patient is generally with the long axis of the tooth. Any traction not in the long axis of the tooth may tilt and loosen the teeth and potentially damage the TMJ, especially when traction is applied for 30-60 minutes, the usual length of most dental procedures.

Moreover, the inflexible handle 32 may allow the patient to adjust the tooth piece 30 from the finger hold 33, which may be difficult or impossible with embodiments that include a flexible member. The patient may also be able to control the tooth piece 30 when closing the mouth at certain intervals to rest and to maintain its position with the teeth. Maintaining the position on the teeth would be extremely difficult with a flexible connection between the hand hold means and the tooth piece, especially without any accessory attachment means to the teeth similar to the grooves or posts previously discussed.

As best illustrated in FIG. 3A, the tooth piece component 30, which fits over the lower teeth, may include a channel 34 with posts 31 on either side. The inflexible extension component 32 may extend out of the mouth and over the lip (not shown) then downward to a point below the chin (not shown.) The lower or caudal portion of this extension component 32 may include (or alternatively may be attached to) the gripping member 33, which may be a handle, a handheld ring, or the like. The tooth piece or mouth piece 30 may be fairly rigid, so as not to slip off the perch on the lower teeth. This rigidity may be important in maintaining the relationship between the mouthpiece 30 and the teeth and in the ability to provide gradual and graded traction on the device 28, without it slipping off the teeth because of flexibility in the tooth piece. Flexibility may preclude the type of graded and gradual gentle pressure needed to accomplish maximum and comfortable mouth opening. The posts 31, or some other similarly functioning element to secure the tooth piece 30 to the teeth, may also provide the necessary stability for the device to function properly. For example, there may be times during a dental procedure when the dentist is not actively engaged in the mouth and changing tools or components that the patient may close or partially close the mouth. During this action of closing the mouth, the tooth piece 30 may become dislodged or malpositioned, if there were no means on the tooth piece 30 to secure it, especially if there was a flexible connection. The flexible connection would preclude control of the tooth piece 30 by the patient and the ability of the patient to adjust the position of the tooth piece 30 at all.

In addition, the device may have a tensiometer interposed between the extension component 32 and the gripping member 33, so that when downward traction is provided by the patient, the tensiometer displays the force. This display may encourage the patient to provide more or less traction by emitting different audible sounds if the tension provided by the patient was more or less than desired. This tensiometer-based alarm may be an important safety feature, which may optimize the opening of the mouth while preventing damage to the temporomandibular joint and other tissues. The tensiometer may be releasably attached to the gripping member 33 or some other point on the handle mechanism, and another traction point or finger hole may be provided from which to provide traction. As well, the tensiometer may be provided separately for employment with any of the mouth opening devices described herein. It may be attached to the component, which extends out of the mouth, a handle, or the gripping member 33 in one of any number of ways.

FIG. 3B is a front view of the mouth opening device 28, illustrating its thin profile. The thin design may occupy less space than thicker design while maintaining strength necessary to accomplish the necessary tasks. The tooth piece 30 may contain a swivel mechanism at the connection to the handle so that the tooth piece can be turned in relationship to the handle so that the handle can be positioned at virtually any position from one side of the mouth to the other.

FIG. 3C is a bottom-up view of the device 28, slightly tilted to demonstrate the finger loop 33 is directly beneath the tooth piece 30.

FIG. 3D is a bottom view of the tooth piece 30, demonstrating the position of the posts 31. This arrangement may allow the device 28 to be positioned in the midline or ninety degrees from midline while still positioning the finger loop 33 directly under the front teeth and allowing the downward traction to be along the long axis of the teeth.

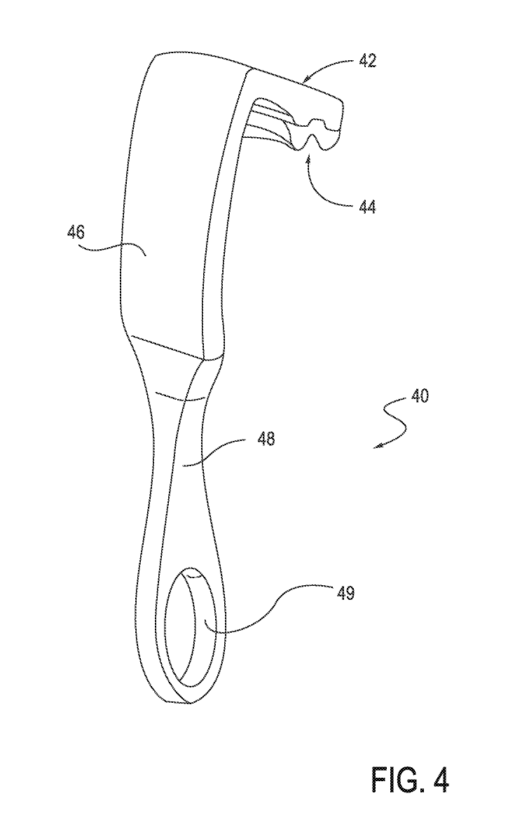

FIG. 4 is a posterior perspective view of a mouth opening device 40. The components of the mouth opening device 40 may be the same or similar in many respects to that of the other mouth opening devices described herein and vice versa. The device 40 may include a tooth engaging portion 42, which may include a soft, tooth-contacting surface 44, a neck portion 46 and a handle 48, which may include a finger hole 49. The top or apex of the neck portion 46 may not protrude as far cephalically in some of the other mouth opening devices described herein.

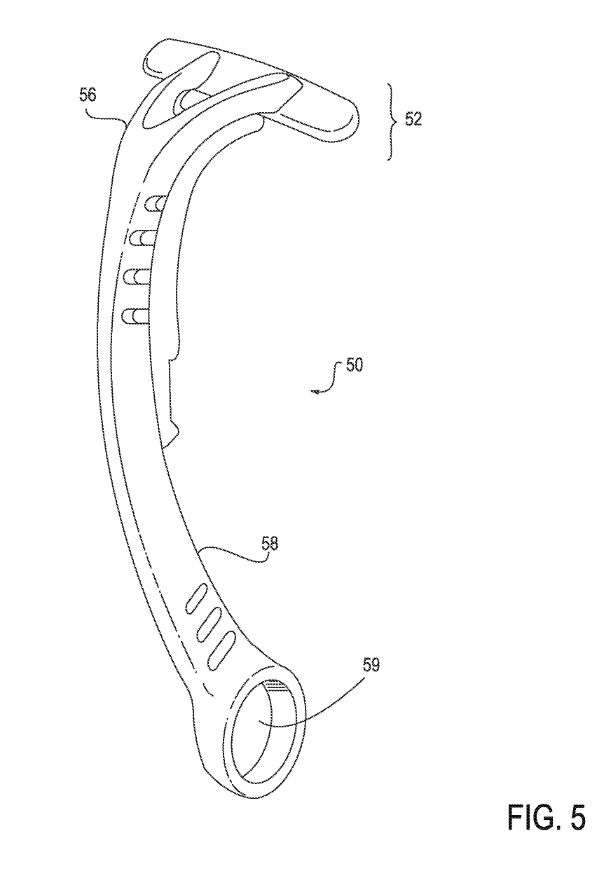

FIG. 5 is a perspective view of a mouth opening device 50, which may include a tooth engaging portion 52, a neck portion 56, and a handle 58 with a finger hole 59. The components of the mouth opening device 50 may be the same or similar in many respects to the other mouth opening devices described herein and vice versa. The tooth piece 52 may include any of the tooth-engaging components described herein, such as the posts and troughs described herein. As well, because of their low profile design, the more cephalic aspect of the handle may provide a finger rest for the dentist to assist in stability of the dental devices and hands and fingers within the mouth. The direction of force may be with the long axis of the teeth.

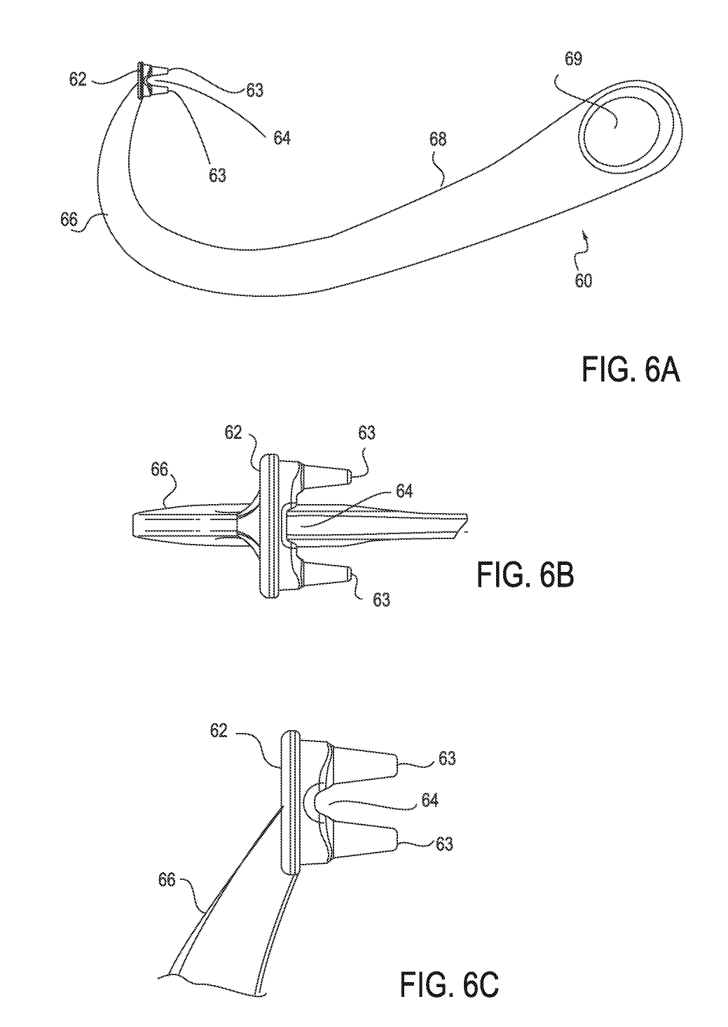

FIGS. 6A-6C are side, close-up front and close-up side views, respectively, of a mouth opening device 60. The components of the mouth opening device 60 may be the same or similar in many respects to that of the other mouth opening devices described herein and vice versa. The device 60 may include a tooth engaging portion 62, a neck portion 66, and a handle 68, which includes a finger hole 69. The tooth engaging portion 62 includes multiple posts 63, which are typically made of a softer material than the rest of the device 60, and which form a bidirectional trough 64 (or “groove”). The apex of the neck portion 66 may be less prominent, projecting not as far cephalically as in the mouth opening device 5 described herein. The curvature of the handle neck portion 66 and handle 68 may be exaggerated compared to that in the mouth opening device 5 described herein, which may provide additional room for the mandibular body and associated soft tissues within the curvature, so the device 60 can be placed ninety degrees from midline while still engaging the middle front teeth. The distance from the front teeth to the side of the jaw may be greater than the distance from the front teeth to the mental protuberance or chin, which may be appropriate for the device 60 if used from the side while the tooth piece is engaged over the lower middle front teeth. This aspect of the construction of the device gives the device 60 may give more utility than device which must be applied and used only from a midline position as it positions the device 60 to one side and out of the dentist's way.

FIGS. 6B and 6C are close-up views of the tooth piece 62, demonstrating the combination of the shallow groove 64 and posts 63 that may accommodate the front teeth from a midline position or ninety degrees from midline. A groove alone may not accommodate all different configurations of lower front teeth and different sized mandibular arches that occur in the general population. The variations in anatomy may demand different sized and shaped grooves if the tooth piece was constructed only with a groove or trough to engage the teeth. The combination of the groove 64 with the posts 63 will provide the stability and secure engagement necessary for the device to provide adequate downward traction without slippage. The tooth piece 62 may also be constructed with a soft durometer material, at least in the trough or groove 64, which may allow the teeth to indent that material when engaging the device, creating traction and more stability than would be present if the material was not soft and compressible. Hence, the tooth piece 62 may comprise at least one or a combination of the following: one or more grooves 64 or troughs to receive the teeth, posts 64 or other means to receive the teeth, and a softer durometer material at the base.

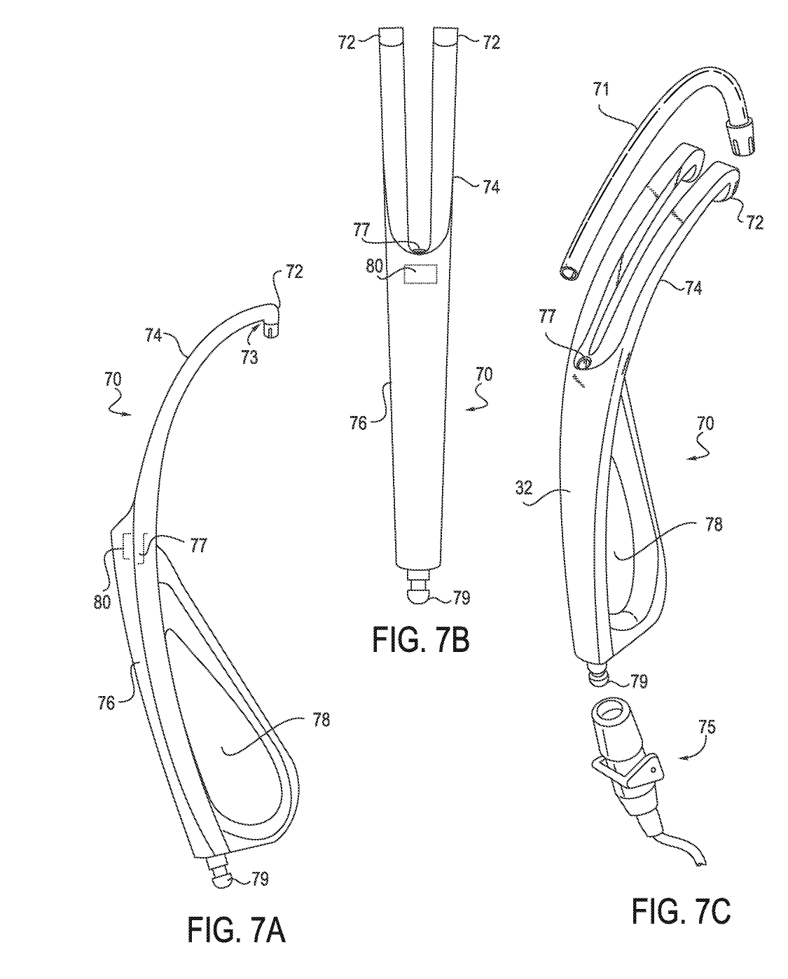

Referring now to FIGS. 7A-7C, a mouth opening device 70 may include suctioning component(s) to provide suction through the device 70. The components of the mouth opening device 70 may be the same or similar in many respects to that of the other mouth opening devices described herein and vice versa. This combination of comfortable distraction of the jaw and suction of saliva may obviate the need for recurrent interruptions of the procedure to aspirate saliva from the mouth. In some procedures, it may even obviate the need for a dental assistant. It generally will make it simpler for the patient to hold onto only one item.

Suction or evacuation may be provided by either attaching a standard or modified suction device to one of the mouth opening devices described herein, or alternatively or in combination, may be provided via a built-in suction channel within a handle of the device. The device 70 may include a tooth engaging portion 72 with an angled section 73, a neck portion 74, a handle 76, which may include a hand hold 78, a suction channel 77 in the handle 76, and a suction connector 79 at the caudal end of the handle 76. As illustrated in FIG. 7C, a piece of suction tubing 71 may be connected to one end of the suction channel 77, and a source of suction force 75 may be attached to the suction connector 79, in order to provide suction within the mouth during a procedure.

FIGS. 7A-7C illustrate a mouth opening device 70 that includes a suction component. The tooth engaging portion 72 (or “tooth piece”) may be configured as a rounded hook and includes a sharp corner 73, to engage the lower front teeth, instead of the posts and grooves described earlier. Alternatively or in combination, the posts and grooves may be employed in the mouth opening device 70. The neck portion 74 extends from the tooth engaging portion 72, and the handle 76 with the hand hold 78 extends from the neck portion 74. As illustrated in FIGS. 7B and 7C, the tooth engaging portion 72 and the neck portion 74 may be forked, so that each portion has two halve or prongs. The suction channel 77 in the handle 76 may extend from a suction tubing recess 80 at one end to the suction connector 79 at the other end.

FIG. 7B is a frontal view of the device 70, which demonstrates the forked neck portion 74 and tooth engaging portion 72. Between them may be a space for the suction tubing. At the junction of the neck portion 74 with the handle 76, the suction tubing recess 80 may be configured to receive a proximal end of a piece of suction tubing 71, preferably with a friction fit. It may comprise an 0 ring or other means to secure within the cavity or recess 80. Alternatively or in combination, a ribbed connector may be used instead of the cavity or recess 80.

FIG. 7C is an oblique perspective view of the device 70, which illustrates how a standard suction tube 71 may be inserted into the cavity or recess 80, so that the tip of the suction tube fits over the front teeth adjacent to the tooth pieces 72. With the suction tube 71 in place and the caudal end of the device connected to a suction hose 75, the suction tube 71 is stabilized, while downward traction can be provided by the patient.

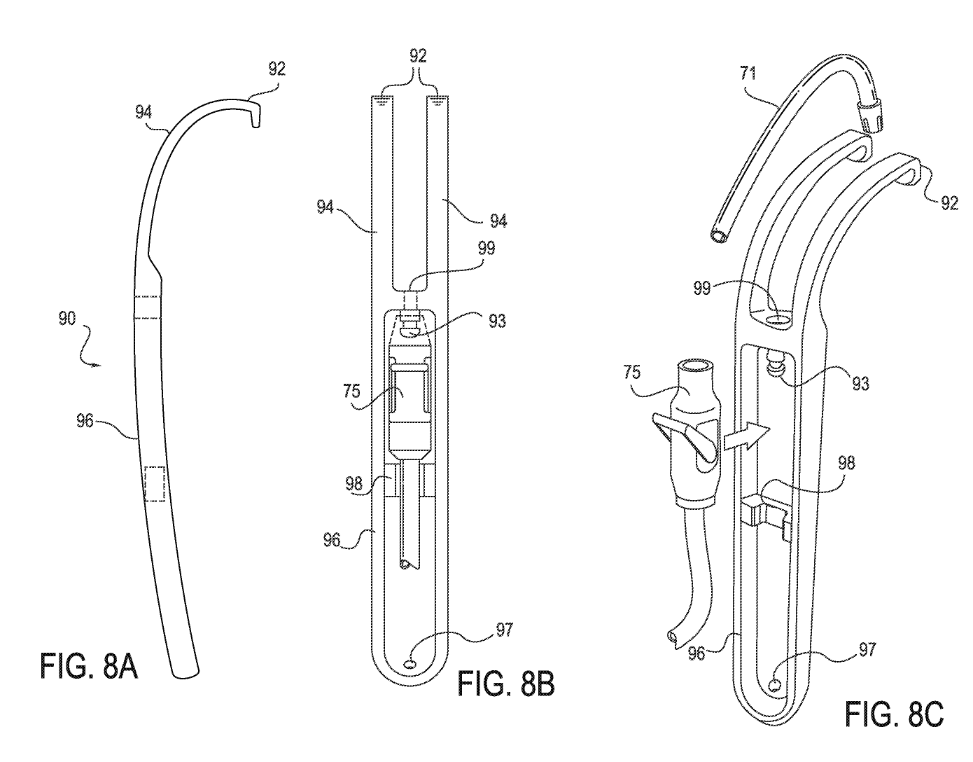

FIGS. 8A-8C are side, front, and rear perspective views, respectively, of a mouth opening device 90. The components of the mouth opening device 90 may be the same or similar in many respects to that of the other mouth opening devices described herein and vice versa. The device 90 may include a tooth engaging portion 92, a neck portion 94 and a handle 96. The tooth engaging portion 92 is forked and has a sharp corner to secure the teeth and a very low profile overall. The neck 94 may also be forked. The device 90 may also include a suction tube recess 99 and a suction valve connector 93, which are configured to attach to suction tubing 71 and a suction hose 75, respectively. The handle 96 may include a tube rest 98 for supporting the suction hose 75 and a finger hold 97 for facilitating holding of the handle 96 by the patient.

FIG. 8C is an oblique perspective view of the device 90, with the suction tube 71 and suction hose 75 detached. Standard suction tubes and standard suction valves that are commercially available may be used with any of these embodiments. Alternatively or in combination, specialized tubes and connectors may be used with any of the mouth opening devices described herein.

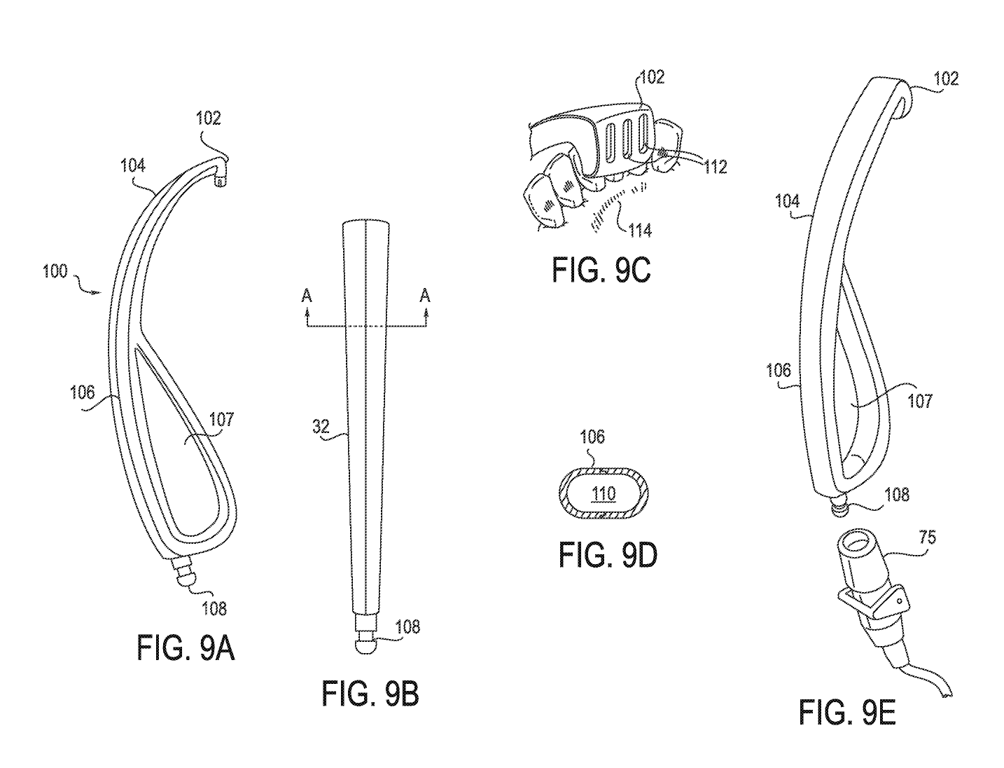

Referring now to FIGS. 9A-9E, a mouth opening device 100 may provide suction without the use of an additional suction tubing piece. The components of the mouth opening device 100 may be the same or similar in many respects as the other mouth opening devices described herein and vice versa. The device 100 may include a tooth engaging portion 102, a neck portion 104, and a handle 106, which includes a hand hold 107 and a suction connector. As illustrated in the cross-sectional drawing of FIG. 9D, the handle 106 and the neck portion 104 may both be hollow, thus forming a suction channel 110, which extends from the tooth piece 102 to the suction connector 108. As illustrated in FIG. 9C, suction inlets 112 into the suction channel 110 may be located along one or more sides of the tooth engagement portion 102. The suction inlets 53 may even extend even further toward the gingival margin 114 than is indicated in this illustration, even curving under and abutting the gingival margin 114 in some cases. With this configuration, the device 100 can help evacuate the mouth of saliva, if the patient closes his/her lips about the distal aspect of the device 100.

Referring to the tooth piece 102, it may be desirable that the tooth piece 102 extend as far toward the gingival margin as possible so that suction is optimized, but without contacting or impinging on the gums or gingiva. This could range from 3 to 15 mm, but optimally may be 6-9 mm. Since the suction inlets 112 may not extend to the dependent portion of the mouth where saliva and fluids collect, the mouth may be closed and the lips pursed around the neck portion 104 to evacuate fluid from these dependent portions of the mouth. In this position, with the lips closed around the neck portion 104, air may travel through the nostrils, the nasopharynx, and the oropharynx and out through the suction inlets 112, and, in the process, will evacuate fluid and saliva that may be pooled in the dependent portions of the mouth.

Alternatively or in combination, the inflexible one-piece construction of the handle 106, the neck portion 104 and the tooth piece 102 may allow the device to be tilted upward by elevating the handle 106 so that the tooth piece can be directed into dependent portions of the mouth by the patient or the dental practitioner to directly aspirate and evacuate fluid and saliva. A compact tooth piece 102 may allow the positioning the suction inlets 112 in a variety of areas within the mouth that would not be practical if the tooth piece 112 were larger. Moreover, without the inelastic, inflexible neck portion 104, this maneuver may not be possible. As well, the surface of the neck portion 104 which abuts the upper and lower lips may be smooth for this maneuver to be practical as the device may be more or less inserted into the mouth. Irregularities or sharp corners could irritate or even lacerate the lips during this maneuver.

Alternatively or in combination, a simple attachment mechanism may be provided on the handle proximal to the tooth piece to releasably or fixably attach the suction component and the attachment mechanism may be one or more of metal, rubber, plastic, polymer, fabric, fiber or adhesive or the like. Moreover, the intraoral suction component the mouth opening devices may be positioned so that there is tubing or other similarly function element to provide suction that may be placed over the front teeth or, alternatively or in combination, along the labial or front side of the teeth and circle dorsally around the molars so that the tip of the suction component is positioned on the lingual or back side of the lower front teeth. This portion of either suction component may also comprise a component which serves to displace at the cheek and/or the tongue away from the area in which the dentist intends to work. In other words, a displacement component may be combined with the suction component and may be placed either over the lower front teeth or along the labial or front side of the teeth and then around the molars so that it provides both suction and displacement. This displacement of the tissues may usually be away from the ipsilateral side occupied by the suction component. The displacing component of this displacing suction component may be tubular, flat, or any other shape which provides both suction and displacement and may provide such functions in a relatively low profile. This may provide exposure for the dental practitioner and keep the moist tissues of the cheek and tongue away from the area being treated.

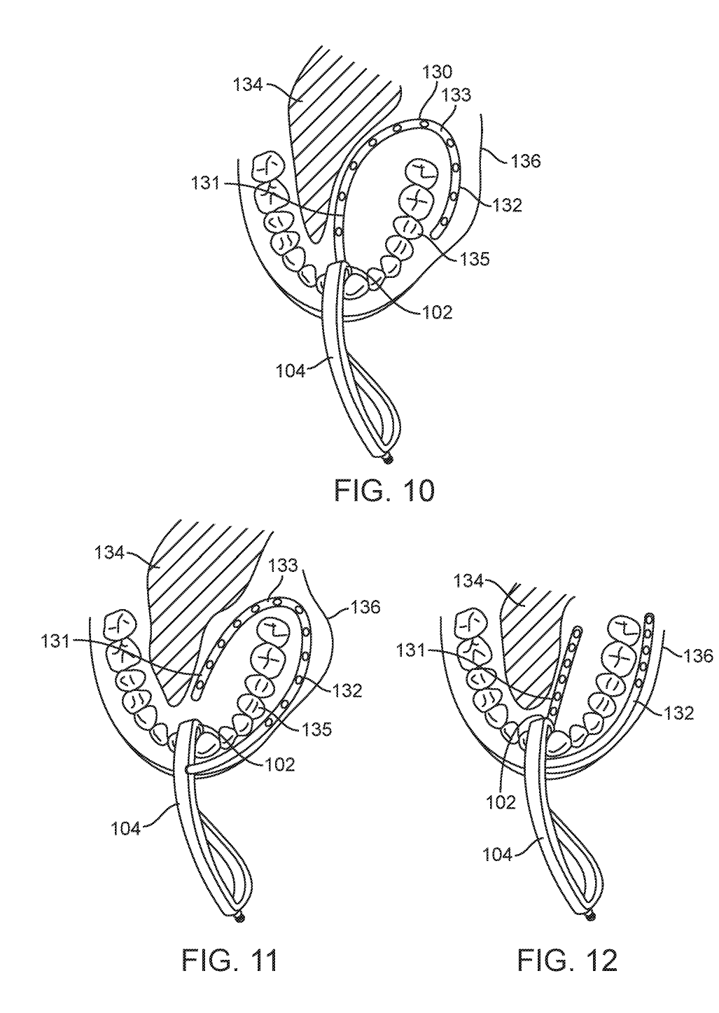

An example of the device is shown in FIG. 10. The displacement mechanism is shown as attached to the device 90, for example, but it may be attached, affixed to or connected to any device which distracts the jaw downward and provides suction. In the devices in which the suction/distraction component is placed over the lower front teeth, as shown in FIG. 10, the suction/distraction component 130 may comprise a lingual component 131 adjacent to and displacing the tongue 134 and a labial component 132 which may be placed between the teeth 135 and the cheek 136 and may displace the cheek 136 away from the teeth 135.

The position of the suction and the displacing means may vary, depending on whether the device is placed so that it exits the mouth midline or on the right or left side and whether the side in which the device is placed is the ipsilateral or contralateral side of the mouth in which the dentist is working. Hence, multiple different configurations of the suction and displacement components are likely, as well as the attachment component to the suction and whether the suction is integral within the handle or a separate tubing, which may be attached to the device and on which side it is attached or connected to the handle.

In FIG. 11, the suction/displacement component 130 is attached to the distal aspect of the neck 104 of the traction device preferably on either side, but also possibly underneath or over the distal neck 104. The labial component 132 may comprise the proximal portion of the suction/displacement component 130 and may displace the cheek 136 while providing suction in that region. The lingual component 131 may comprise the distal portion and may provide displacement of the tongue 134 and suction in the floor of the mouth. Both may be tubular structures as illustrated with holes 138 to allow the fluids to enter and be removed.

In FIG. 12, the lingual portion 131 of the suction/displacement component 130 may originate from the tooth engaging portion 102 of the mouth opening device and the labial component 132 may originate from the distal neck 104 of the mouth opening device. The suction/displacement component 130 may be fixed to or realeasably attached to the tooth engaging portion 102 or the handle 104 of the device in any number of ways, and the components may be attached to any of the different elements of the mouth opening devices described.

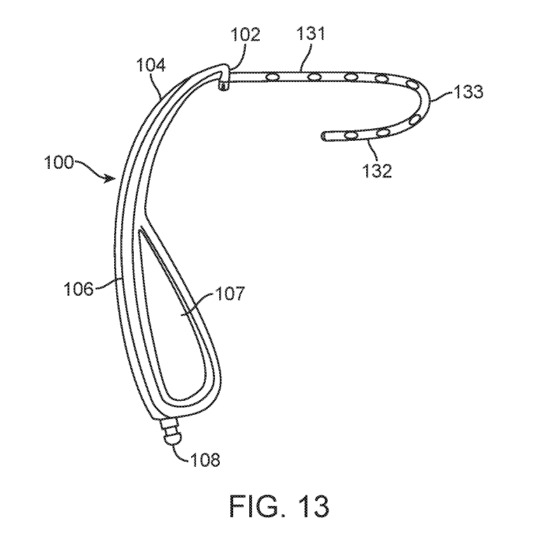

In FIG. 13, the proximal portion of the suction/displacement component 130 is shown as attached to the tooth engaging portion 104, the lingual portion 131 being proximally positioned and the labial component 132 positioned distally.

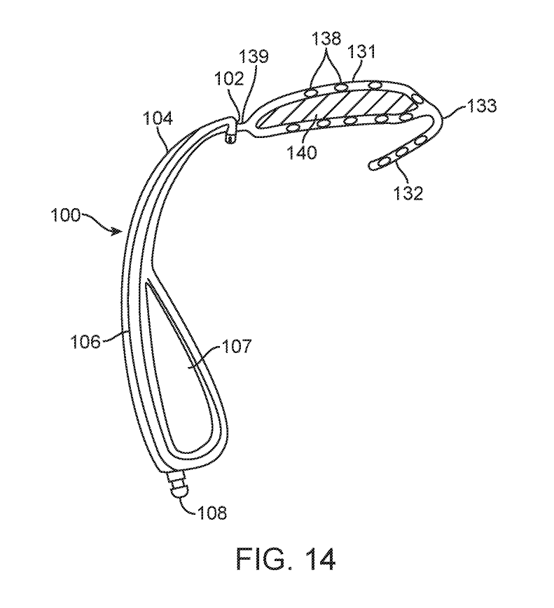

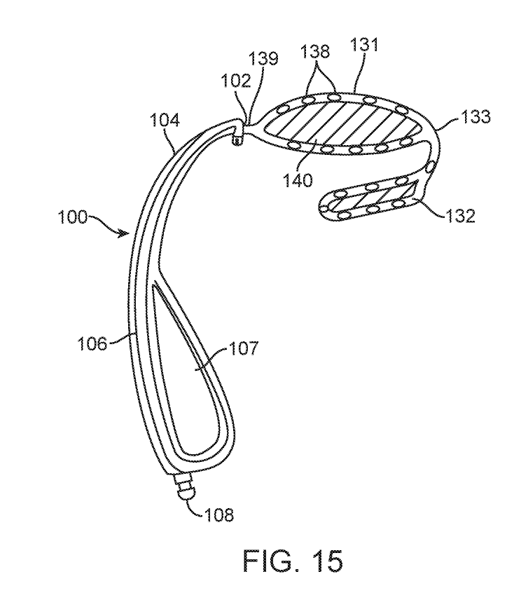

FIG. 14 demonstrates a similar configuration as FIG. 13, but the lingual component 131 may be wider than just a tubular structure. The lingual component 131 may comprise two tubes with holes 138 and a membrane 140 between the two tubes to keep them spatially separated. The membrane may be plastic or any one of a number of materials. Smaller tubes may even extend into the membrane 140 to provide additional suction to the lingual 131 and labial portions 132. Point 133 may be where the device wraps around the posterior molar and where the lingual portion 132 is directed forward in this and other embodiments herein. As shown, the membrane 140 is between the tubular structures, but it may extend beyond the margins of the tubular structures from 1 mm to 3 cm. Moreover, the membrane 140 may be constructed of or comprise a reflective material to enhance the available light within the mouth.

FIG. 15 demonstrates a configuration similar to that shown in FIG. 14, but the labial portion 132 may be a double tubular structure with a membrane 140 between the tubular structures. The point 133 may be a singular structure as shown to facilitate the curve around the posterior molars, but may be a double tubular or other structure (not shown) as well.

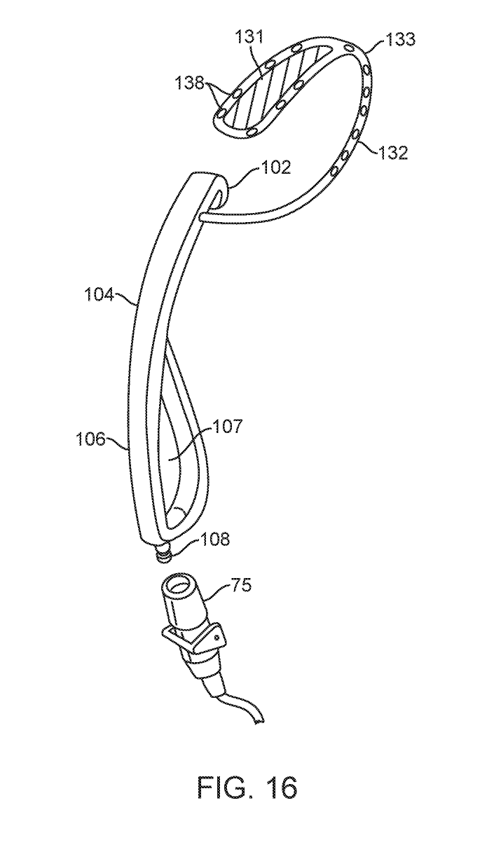

FIG. 16 is a perspective of a mouth opening device configured similarly to that shown by FIG. 11. The labial component 132 is proximal and the lingual component 131 may be distal. It may be attached to any one of a number of points of the distal neck 104 of the device. Here it is attached to the side. The labial component 132 may displace the cheek and the lingual portion may displace the tongue.

The construction of suction/displacement component 130 may utilize any one of a number of materials including plastic, silicone, plyethylene, C-Flex, plyurethane, Chonoprene amongst others. A material of a durometer that maintains some stiffness, but may be flexible enough to be comfortable may be used. In the one piece configuration, it may be generally horse shoe shaped and may have a relatively low profile area 133 in the turn between the lingual 131 and labial 132 portions.

As illustrated herein, suction can be provided in a number of configurations, and thus, the described are merely examples of some of the configurations. Suction may be provided by just the suction tube, suction valve, and suction hose, or by providing connections between some of these suction components. The suction components also may attach to the elements of a device, to stabilize the suction apparatus. The elements of a device may stabilize the suction components by cradling the suction components, for example, rather than being connected or attached to them.

In any of the devices described herein, an added part or component may be provided to prop the mouth open. The devices of the present disclosure may be intended to keep the mouth open as described, but may not prevent the patient from closing the mouth unexpectedly because of a sudden pain or impulse to close. Abrupt closing could potentially displace a drill, burr, or other dental device and may cause damage to the instrument, the patient, and/or the dentist or dental practitioner. Ideally in at least some cases, this propping mechanism may not actually prop the mouth open, but may only serve to keep the mouth from closing significantly. In other words, it may function as a safety mechanism more than a means to keep the mouth open. The teeth may not abut this component, unless and until the patient unexpectedly closes the mouth. For example, the mechanism may simply comprise a material added to the cephalic aspect of the tooth piece or may be a separate component which is releasably or fixedly attached to the distal aspect of the handle adjacent to the tooth piece. Alternatively or in combination, these elements to prop open may be attached to the suction component or may be separate.

The devices described herein may also be used in concert with other commonly used dental devices, including dental dams and other devices. One may combine the current device(s) with a displacement device that displaces the tongue and cheek away from the targeted teeth. This may be accomplished, for example, by modifying the suction component as described herein to accommodate the displacement device and suction. One may also combine the mouth opening devices described herein with a lip retractor configured to retract the lips to gain better exposure to the oral cavity.

Additionally, a light or means of illumination may be added to the handle, the tooth piece, the suction component, or the displacement component, or it may be provided separately and attached to any of the components herein. Moreover, any of the components may be constructed of reflective material, which may serve to illuminate the teeth and oral cavity by reflecting light from the means of illumination described herein or from some other source of light. Even further, any of the components, and especially the displacing component, may be constructed of material that glows or emanates light when that substance contacts moisture or glows or emanates light because of some other physical or chemical reaction. This process could be because of fluorescence, chemoluminsecence, phosphorescence, light emitting diodes, or even organic light emitting diodes, among others. Any combination of light emitting substance and reflecting substance may be used.

The mouth opening device(s) described herein may also be made more visually attractive, which may be especially important when used in children. The mouth opening device(s) may comprise a detachable structure, a structure or attachment fixed to the device, or art applied to the device in one of several manners. While this visual component may represent an animate or inanimate object, one such structure may comprise a face or portions of a face that attaches to the neck to give the device a personality. This visual component may comprise one or more of eyes, mouth, ears, or nose and may represent an animal, person, plant, cartoon, or other figure. The visual component may be attached to, applied to, or incorporated into the neck or handle of the device. An exemplary visual component may be configured so that it may be removed from the device, preferably at the end of the procedure, and given to the patient. The visual component may be constructed so that it may be worn by the patient on clothing, in the hair, or even as a ring on a finger.

In maintaining a mouth of a patient in an open position during a dental procedure, methods of using the mouth opening device(s) described herein may comprise placing, by either the patient or the dental practitioner, a groove of a tooth engaging portion of a mouth distraction device over at least one lower tooth in a lower jaw of the patient's mouth and pulling down or providing traction on a handle of the mouth distraction device, preferably by the patient, to maintain the lower jaw in an open position relative to an upper jaw of the patient's mouth. This traction may be substantially continuous or intermittent. Suction may be provided by attaching a suction or vacuum source to or incorporating the suction or vacuum source within the distraction device. A tongue, cheek, or a tongue and cheek displacement element may be positioned by the dental practitioner to displace the tongue and/or cheek away from the area to be treated. A lip retraction element may also be positioned by the dental practitioner.

In at least some cases, optimally, the patient would provide downward traction on the mandible that would be sufficient to open the mouth as wide as possible while avoiding discomfort. The ideal balance of degree of distraction and the lack of discomfort can be achieved by the patient controlling the downward force of traction over the course of the procedure. The degree of distraction may be varied by the patient over the course of the procedure and may gradually increase as the initial traction causes the muscles of the jaw to relax. This variation may cause the jaw to open even more as the procedure progresses. The patient may intermittently close the mouth to relax, swallow or rest or may purse the lips about the neck of the device to evacuate the fluid within the mouth. Traction then may be reapplied by the patient to a degree that opens the mouth as wide as possible without generating discomfort. Repeating the traction, relaxation, and traction cycles may allow the mouth to open even further and without discomfort. By providing a handle that is rigidly affixed to the tooth engaging apparatus, the devices may maintain their relationship and prevent the tooth engaging portion from becoming displaced from the teeth when the patient closes the mouth to rest, relax, swallow, or evacuate fluid as may be the case with prior art devices. At the termination of the procedure, the device may be removed by the patient or the dental practitioner and properly disposed.

Special mention of the ability to evacuate saliva is in order. Standard saliva ejectors are plastic or metal tubes with a cap on the end in which there are slits for the saliva, water and other fluids to enter. These saliva ejectors are connected to suction tubes and eventually to a suction machine. They function moderately well but frequently the suction slits are adjacent to or touch soft tissue and oral mucosa, and the suction causes the soft tissue or mucosa to be sucked into the suction slits. This is uncomfortable and can damage the sensitive and delicate mucosa and soft tissues of the oral cavity. To prevent this, a dental assistant is needed to administer the suction intermittently when needed and to prevent the “grabbing” of mucosa and soft tissue by the suction tip. This adds unnecessary expense and complexity to the procedure as frequently the hands of the dentist and the dental assistant are all within or near the mouth. It is an object of a particular embodiment of the current invention to provide an atraumatic suction device which may be used as a saliva ejector that does not “grab” the mucosa or soft tissue, and provides more or less continuous suction, obviates the need for a dental assistant to provide suction and is comfortable and formable at the same time.

Some of the embodiments of the current mouth opening invention (FIGS. 1-9) may not need an improvement in the suction design as the suction holes or apertures are directly over the lower incisors in most cases and not near mucosa that could be inadvertently sucked into the slits or holes of the device. However, alternative embodiments, such as FIGS. 10-16, which have tubular structures extending to the posterior oral cavity, alongside the tongue, near the cheeks or into the tonsillar fossae may indeed tend to aspirate tissue along with fluids, especially if a high volume ejector (HVE) is utilized. Hence, in these instances without a dental assistant to provide and monitor the suction, protecting the tissues of the mouth while providing HVE suction is critical to keep from damaging the soft tissues. In the dental hygiene arena, ultrasonic scaling has recently become widely used to clean the teeth. This process uses more water than other procedures, mainly to dissipate the heat generated by the device. There is a need for the hygienist to evacuate the copious amounts of water in the mouth from this technique without an assistant present and to do so without grabbing or sucking tissue with the HVE suction.

Two other elements to consider are comfort and formability. The apparatus must be comfortable or it will not be tolerated by the patient, but at certain, but not all, times must be formable so it can be shaped for the individual patient's mouth or placed on the contralateral side. Formable elements are usually stiff and uncomfortable and frequently have a metal wire to provide stiffness, whereas comfortable tubes are usually soft but non-formable. Hence, there is a dilemma in providing a safe and comfortable saliva ejector or dental suction apparatus that solves all of the issues above in one device.

The accessory suction device of the current invention solves all five of these issues: 1) Protects the mucosa of the mouth during suction of saliva ejection, 2) Does not need an assistant to provide and monitor suction, 3) Is comfortable, and 4) Is formable into different shapes and lengths and 5) Is capable of evacuating large amounts of fluid from the oral cavity. This is accomplished by providing an inner suction tube that is firm and formable but is coaxially placed within a tubular mesh braid which provides the soft comfort that the inner suction tube does not. More importantly, the tubular braid displaces the tissues and mucosa away from the suction slits or holes of the suction tube and prevents the aspiration or sucking of tissue or mucosa by the inner tube which provides the actual suction. The inner tube is rather stiff and formable, but the tubular mesh braid covering it is soft and pliable causing the net result to be a comfortable configuration that will be readily accepted by the patient but one which can be modified for each particular patient and is capable of evacuating large volumes of fluid.

The tubular braid is preferably formed as a mesh of individual non-elastic filaments (or “yarns”), although it may have some elastic filaments interwoven to create certain characteristics. The non-elastic yarns can be materials such as polyester, PET, Polypropylene, polyamide fiber, composite filament wound polymer, extruded polymer tubing, stainless steel, Nitinol, or the like so that axial shortening causes radial expansion of the braid. These materials have sufficient strength so that the braid element will retain its expanded condition in the mouth while protecting the tissue from the inner suction tube shaft and the holes and/or slits in that piece as well as cushioning the pressure from the stiff and uncomfortable inner suction tube.

The braid may be of conventional construction, comprising round filaments, flat or ribbon filaments, square filaments, or the like. Non-round filaments may be advantageous to decrease the axial force required for expansion to create a preferred surface area configuration or to decrease the wall thickness of the tubular braid. The filament width or diameter will typically be from about 0.5 mils to 25 mils, usually from about 5 to 10 mils. Suitable braids are commercially available from a variety of commercial suppliers.

The tubular braids are typically formed by a “Maypole” dance of yarn carriers. The braid consists of two systems of yarns alternately passing over and under each other causing a zigzag pattern on the surface. One system of yarns moves helically clockwise with respect to the fabric axis while the other moves helically counter-clockwise. The resulting fabric is a tubular braid. Common applications of tubular braids are lacings, electrical cable covers, “Chinese hand-cuffs” and reinforcements for composites. To form a balanced, torque-free fabric (tubular braid), the structure must contain the same number of yarns in each helical direction. The tubular braid may also be pressed flat so as to form a double thickness fabric strip. The braid weave used in the tubular braid of the present invention will preferably be of the construction known as “two dimensional, tubular, diamond braid” that has a 1/1 intersection pattern of the yarns which is referred to as the “intersection repeat.” Alternatively, a Regular braid with a 2/2 intersection repeat and a Hercules braid with an intersection repeat of 3/3 may be used. In all instances, the helix angle (that being the angle between the axis of the tubular braid and the yarn) will increase as the braid is expanded. Biaxially braided fabrics such as those of the present invention are not dimensionally stable. This is why the braid can be placed into an expanded state from a relaxed state (in the case of putting it into the compressive mode). When put into compression the braid eventually reaches a state wherein the diameter will increase no more. This is called the “Jammed State.” Much of the engineering analysis covering braids are calculated using the Jammed State of the structure/braid. These calculations help one skilled in the art to design a braid with particular desired characteristics. Further, material characteristics are tensile strength, stiffness and Young's modulus (elasticity) amongst others. In most instances, varying the material characteristics will vary the force with which the expanding condition of the tubular braid can exert radially. Even further, the friction between the individual yarns has an effect on the force required to compress and un-compress the tubular braid. For the present invention, the desired characteristics and function are maintaining enough radial force to displace the inner suction tube away from the tissues of the mouth or displacing the tissues away from the inner suction tube for both patient comfort in tolerating the presence of the device and preventing the aspiration of tissue by the suction inlets. This is best achieved by utilizing the tubular braid in a compressed, but not Jammed State. The Jammed State may reduce flexibility and the interstices of the braid may be diminished so that fluid is impeded from flowing freely through the fully compressed braid. Hence, applying the braid to the inner suction tube in a compressed state which approaches, but does not achieve, the Jammed State is preferable.

Tubular mesh braid can be constructed in any one of many ways. Usually a weave pattern is utilized where each member crosses under an intersecting member and then over the next intersecting member, and so on. If the braid is elongated, it has very little outward radial force, but when the ends are compressed and brought closer together, the tube of braid expands outwardly and does gain force and strength as the members develop an angle that approaches 90 degrees more so than in the elongated configuration. It is in this state, i.e., when the ends of the tube of mesh braid are compressed together, that the braid will gain radial strength and be able to shield the suction slits and holes from tissue or mucosa. The braid is still compressible and flexible, hence, comfortable. It is in this moderately compressed state that the braid will also provide an optimized comfortable cover to the stiff and uncomfortable inner suction tube, although simply applying a relaxed braid coaxially over the inner suction tube may indeed function fairly well.

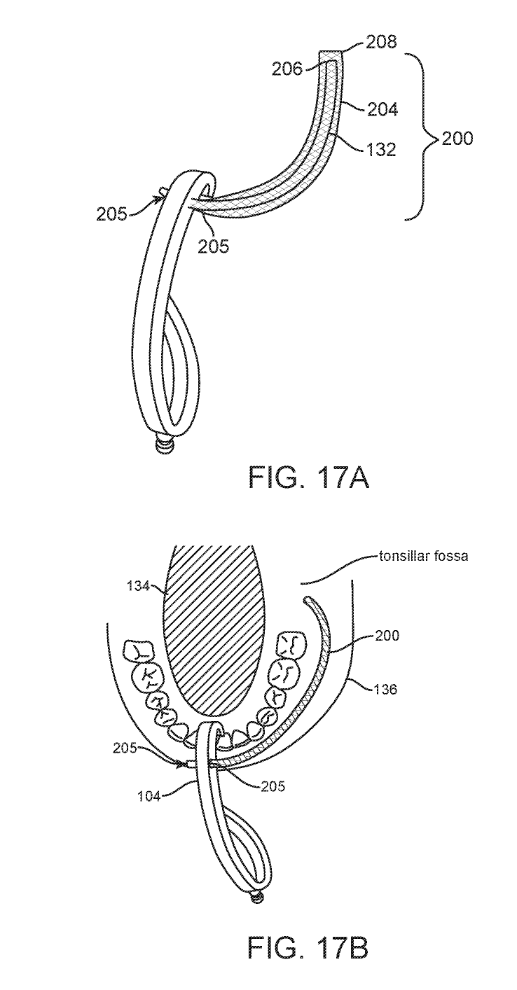

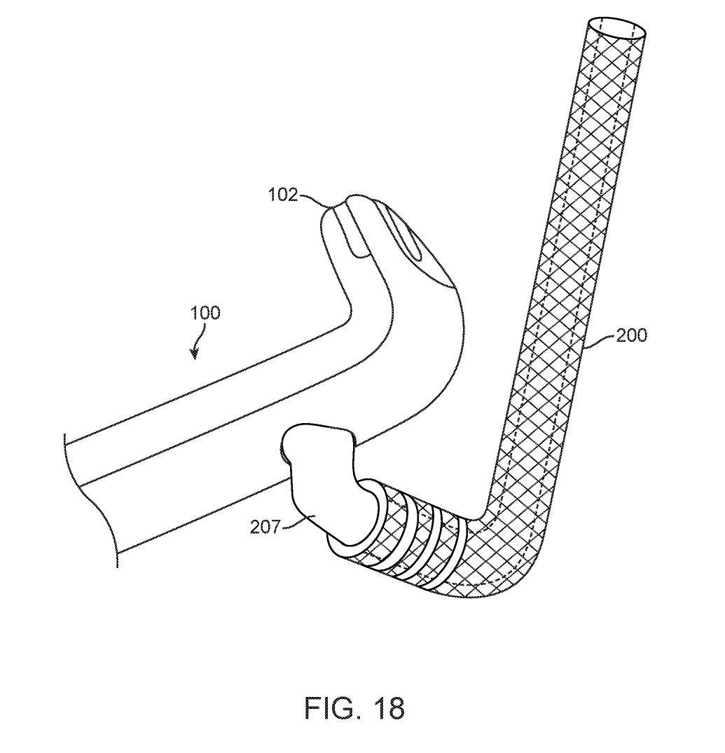

The above coaxial configuration of an inner suction tube and an outer sleeve of tubular mesh braid are collectively referred to as the suction apparatus, and are detailed in FIGS. 17A and 17B. The suction apparatus 200 is attached to the mouth opening device 100 described earlier either from the dorsal or ventral aspect or from one of the sides of that device. The holes or inlets 138 in the tooth engaging portion 102 may be either covered or omitted from the design in the case of alternative suction devices. The suction apparatus 200 may be attached to the mouth opening device 100, which comprises a suction channel within, by means of an attaching mechanism that may take any one of several configurations and may include a simple friction fit of the suction tube apparatus 200 over or within a fitting that is attached to the mandibular traction or mouth opening device 100. In one embodiment the fitting is an elbow fitting 207 as demonstrated in FIG. 18 directed more or less perpendicularly to the axis of the mouth opening device 100 which may be removable and directed to either right or left sides. In other configurations, the fitting may be small nipples 205 as shown in FIGS. 17A and 17B projecting off either side of the mouth opening device amongst other configurations. In this case, suction apparatus may be provided for both right and left sides or, in the case of providing just one suction apparatus, small caps may be placed over the nipple not being utilized. The suction apparatus may also be connected to the tooth engaging portion of the mouth opening device by a fitting (not shown) or a sleeve (not shown) which attaches the suction apparatus 200 to the mouth opening device 100. As well, the suction tube apparatus 200 may be connected to other suction tubes, other devices or may be used as a standalone device (not shown).

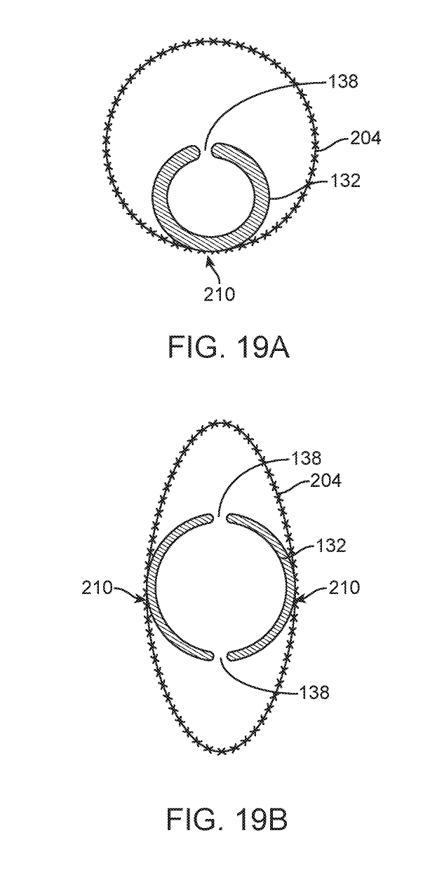

The suction tube apparatus 200 may include an elongate suction tube 312 with holes or slits 138 at least in the distal end, placed inside an expandable tubular braid 204. The tubular mesh braid 204 may extend from the proximal portion of the inner suction tube 132 to at least the distal end 206 of the inner suction tube 132, and in different embodiments may extend past the distal end 206 of the inner suction tube 132. This is illustrated in FIG. 17 in which an inner suction tube 132 is placed coaxially within a more or less compressed tubular braid 204. In this configuration, the suction apparatus 200 is configured to extend into the tonsillar fossa of the oral cavity where fluid frequently collects. If the distal end hole of the inner suction tube is left open, it is important that the tubular braid comprises a section 208 that extends beyond the distal end 206 of the inner suction tube 132 so that it encloses the distal end hole 209 and protects the tissue from being sucked into the distal end hole. To construct the coaxial device, one may compress the tubular braid from end to end and bond the tubular braid to the inner elongate suction tube while the braid is being compressed. The bond may be a heat bond, chemical bond or other means. Bonding the tubular braid to the inner suction tube while the braid is being compressed will give the braid sufficient radial outward force to displace tissue away from the shaft of the inner suction tube hence making the whole structure more comfortable to the patient, and most importantly, keep the tissues away from the slits and holes in the inner suction tube and prevent the aspiration of tissue by the suction. The braid may be bonded to the inner suction tube at or near the proximal end and at or near the distal end, and it may be bonded to the inner suction tube at other locations along the shaft of the inner suction tube. As detailed in FIGS. 19A and 19B, which are cross sections of the improved suction apparatus 200, the bonds 210 may be placed strategically on the device so that the bond 210 is on the opposite side of the inner suction tube 132 from any holes or slits 138 as in FIG. 19A. This would insure that the braid 204 would not approximate the holes or slits 138 and allow any tissue to approximate the holes or slits 138. In fact, the bonds 210 may be arranged on either side of the inner suction tube 132 as in FIG. 19B which would cause the braid to take on an elliptical shape vs. the cylindrical shape.

The foregoing explanation is important in the construction of the current suction apparatus and to achieve the desired goals as simply placing a braid over the inner suction tube or the distal end of the inner suction tube will not achieve the desired goals. Bonding the tubular braid to the inner suction tube in a moderately compressed state is feasible and advantageous. Simple heat bonding of the braid and inner suction tube, or other methods, may be readily utilized for that portion of the braid which overlies the inner suction tube. Heat bonding, chemical, and other methods of bonding are all feasible. It is anticipated that the tubular mesh braid 204 and the shaft of the inner suction tube 132 be related chemically with similar physical properties for facilitate whichever means of bonding is chosen. If a polymer is chosen as the material for the braid and shaft of the inner suction tube, then choosing a material for each from the same or a related polymer family is anticipated to facilitate the bonding process.

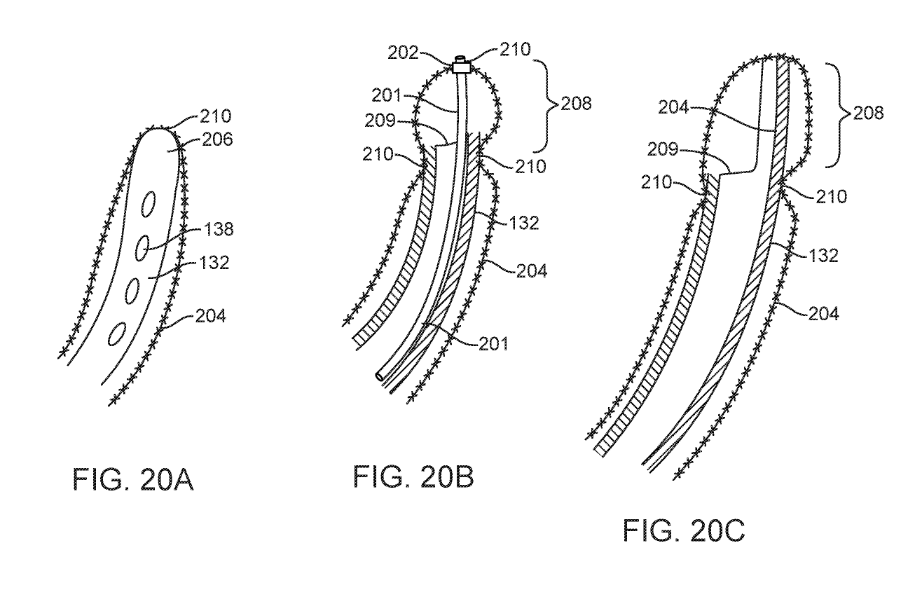

However, extending the tubular braid beyond the distal end of the inner suction tube and maintaining it in a compressed state so that it maintains outward radial forces may be problematic as there is no anchor to maintain the compression of the tubular braid extending beyond the distal end of the inner suction tube. This section of the tubular braid will elongate and loose its radial outward strength or force and not protect the distal end hole of the inner suction tube from sucking the braid and the adjacent tissue into at least close proximity, if not immediately adjacent to, the suction apertures and the distal end hole. To address this dilemma, FIGS. 20A-20C demonstrate potential solutions. FIG. 20A demonstrates that attaching the tubular braid 204 to the distal inner suction tube 132 with a bond 210 of choice, closing the distal end hole of the inner suction tube 132 and placing the suction apertures or inlets 138 proximal to the end 206 of the inner suction tube 132 where they will be protected or covered by the compressed tubular braid is a potential solution. Another configuration shown in FIGS. 20B and 20C that solves this dilemma may be to place and attach an extension member between the distal end of the inner suction tube and the tubular braid end that extends beyond the distal end of the inner suction tube. This member may anchor the distal end of the braid in a compressed state, or at least serve to anchor the braid end in more or less a compressed state hence “protecting” the distal end hole. This extension member may be a separate structure, such as a more or less linear strut that is bonded to both the distal end of the inner suction tube and the end tubular braid securing in a more or less compressed state extending beyond the end of the suction tube. A string like member not shown) that tethers the distal end of the tubular braid to the end of the inner suction tube may maintain the end of the braid in a compressed state and preserve the characteristics needed to keep the tissues away from the inner suction tube inlets. As illustrated in FIG. 20B, the extension member may be an extension of a stiffening wire 201 that may be a part of the inner suction tube 132 to give it formability. The wire 201 creates problems in that it is stiff and may injure tissues. Also the bond 210 to the braid 204 may have to be a metal compression fitting 202 that also may be uncomfortable to the tissues and may cause injury.

As demonstrated in FIG. 20C, the extension member 208 also may be comprised of a portion of the shaft of the inner suction tube 132 that extends beyond the distal end hole 209. Simply removing approximately half or more of the wall of the cylindrical inner suction tube 132 over the distal 1-2 cm or so may provide the extension member 208 needed to anchor the distal braid in a more or less compressed state while effectively placing the distal end hole of the inner suction tube remote from the braid end.

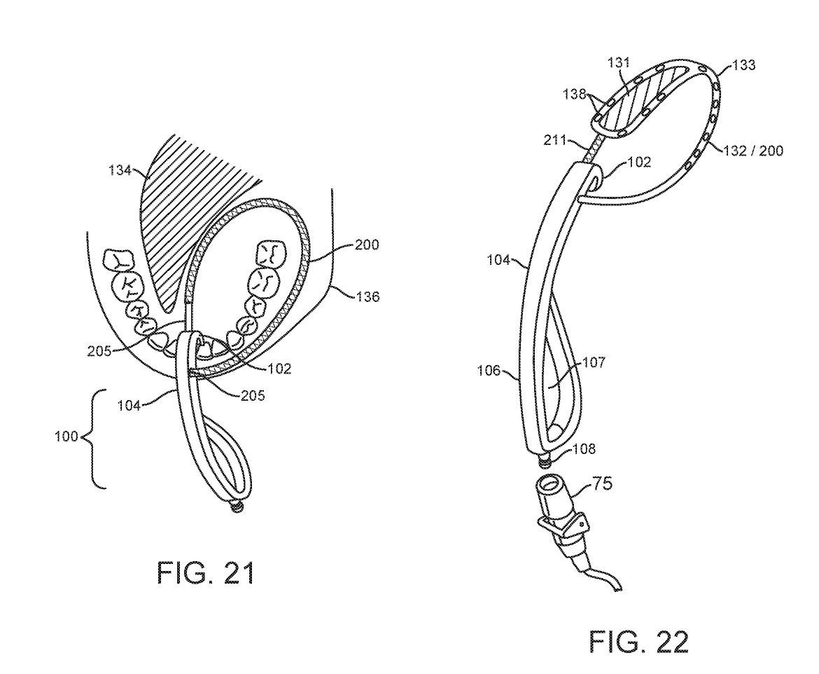

FIG. 21 demonstrates an improvement on the configuration of FIGS. 10-20. The improved suction apparatus 200 described herein is utilized, as it may be with the configurations of FIGS. 10-20, but the suction apparatus 200 of FIG. 21 is connected to the mouth opening device 100 at least two points or locations 205. This serves two purposes: 1) It provides more uniform suction throughout the suction apparatus 200, and 2) It provides stability and strength to the device as it displaces the tongue 134, cheek 136 and other tissues away from the teeth that need instrumentation. These connections 205 may be fittings as described herein, sleeves or alternative fittings or methods, which may be placed on the dorsal or ventral surfaces or sides of the neck 104 of the mouth opening device 100 and may be connected to the tooth engaging portion 102 of the mouth opening device 100 as shown. In this configuration, there may be no need for the embodiments detailed in FIGS. 20A-C.

A method of utilizing the embodiment of FIG. 21 may entail placing the mouth opening device 100 into the mouth and over the lower front teeth while having one end of the improved suction apparatus 200 already attached to the mouth opening device 100. The dental practitioner then would place the improved suction apparatus 200 in appropriate position within the mouth so that it comfortably displaces the tongue 134 and cheek 136 and is positioned for optimal suction. The second end of the improved suction device may then be connected to the mouth opening device 100.

Alternatively, the improved suction apparatus 200 may be utilized in configurations of FIGS. 13-16 in which loops may be formed to better displace the tongue 134 and cheek 136 away from the teeth of operatory interest. In these instances, a second connection to the mouth opening device 100 may be accomplished as demonstrated in FIG. 22 but may or may not provide for suction. The second connection may be for stability only. If this second connection is for stability only, it may take any one of several forms and configurations including a clip or attachment 211 to secure the improved suction apparatus 200 or just the inner suction tube 132 to the mouth opening device 100. This attachment 211 may be adjustable by one of several means so that after comfortably displacing the tongue 134 away from the teeth of interest, the dental practitioner may displace the tongue even more by extending the length of the attachment means 211 and forcing the tongue to the side of the mouth even further hence providing more exposure for the practitioner. The means (not shown) for connecting the attachment mechanism 211 to the suction apparatus 200 and the mouth opening device 100 may be constructed so the tongue displacement apparatus may be purposefully and forcefully positioned a selected and variable distance away from the connection point of the mouth opening device 100.

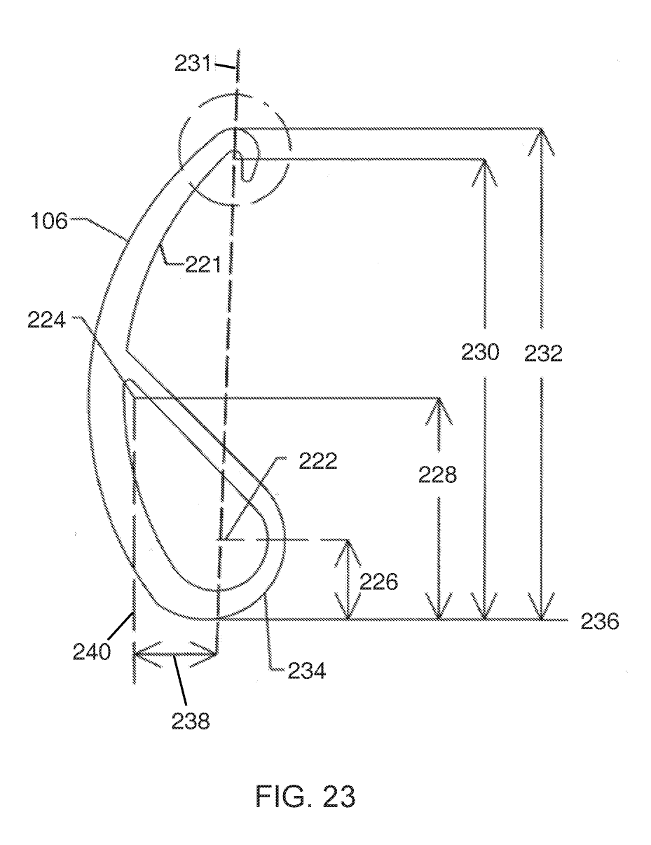

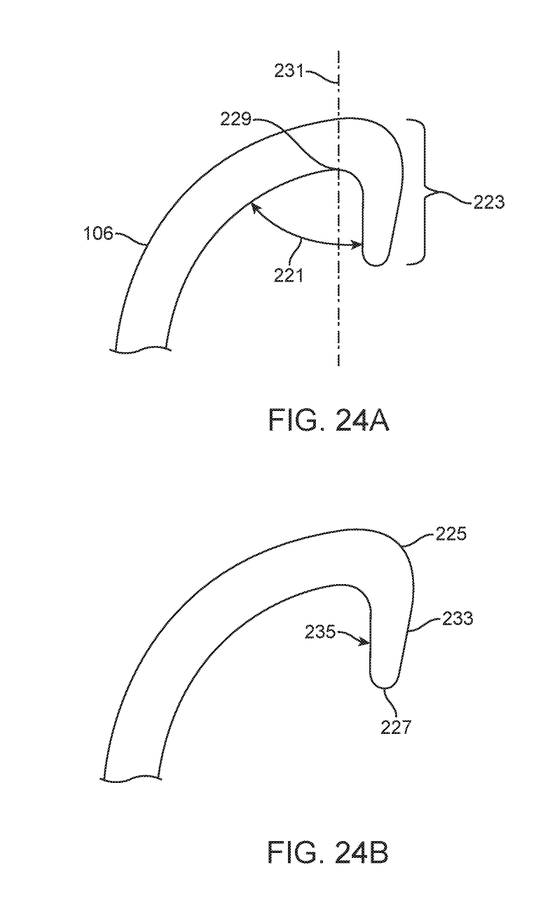

FIG. 23 is an illustration of a handle 106 used to retract the temporomandibular joint. The handle 106 is shown with a pronounced arc which begins at the tooth hook 223 (See FIGS. 24A-B) to and terminates at proximal radius 222. This arc allows for the handle 106 to sweep around the chin and terminate at the proximal handle configuration. 230 defines the arc, when 230 is short it brings the hook to the finger loop diameter 226 closer causing the handle to bulge outward bringing the finger loop 226 closer to the chin causing a unnatural and more uncomfortable situation whereby the hook 223 pulls the tooth out of its natural position. The optimum length for the distance 230 is between 4 and 10 inches. Handle 106 ranges in length 232 from 2.00 inches to 24.00 inches in length from hook to baseline 236. The cutout for the finger engagement is defined by minor radius 224 which is measured from the baseline 236 distally 228 approximately 0.50 inches to 10.00 inches. It is biased off of the longitudinal axis 231 by distance 238 to centerline 240. This length can range from 0.25 inches to 2.00 inches in length. Major radius 222 forms the longitudinal axis 231 with radius 222. Length 226 can range from 0.25 inches to 4.00 inches from baseline 236 measured from its center point. The distance between minor radius 224 and major radius 222 defines the “Tear Drop” shape a shown. For instance the closer the minor radius 224 is to the major radius 222 the more the tear drop becomes a circle. The opposite is true when the minor diameter 224 is further away from the major radius 222 the tear drop becomes more pronounced. In this embodiment the tear drop is formed by minor radius 224 being approximately 0.125 inches and approximately 1.00 inches off of longitudinal axis 231, the major radius 222 being approximately 1.00 inches and on the longitudinal axis 231. The cutout can be used by any finger of either hand but as an example using the thumb the weight of the hand provides enough downward traction as to comfortably relax the temporomandibular joint.

FIG. 24A is an enlarged view of the hook 223. The hook 223 is defined by internal radius 229 which in turn defines angle 221. As an example internal radius 109 can be very acute being 0.020 inches which in turn translates into approximately 25 degrees of engagement from the tooth face 235 which is parallel to longitudinal axis 231. This allows for a very tight purchase on the lower mandible, engaging one or more teeth. The angle 221 can range from 5 to 110 degrees but is preferably between 30 and 90 degrees. The hook 223 length is defined by radius 225 plus length 223 terminating at radius 227.

FIG. 24B is an enlarged view of the hook 223 with tooth face 235 which engages one or more teeth's inner surface. On the hook 223 is a radius 227 at its distal tip which is provided to mitigate irritation to the tissue of the gum.

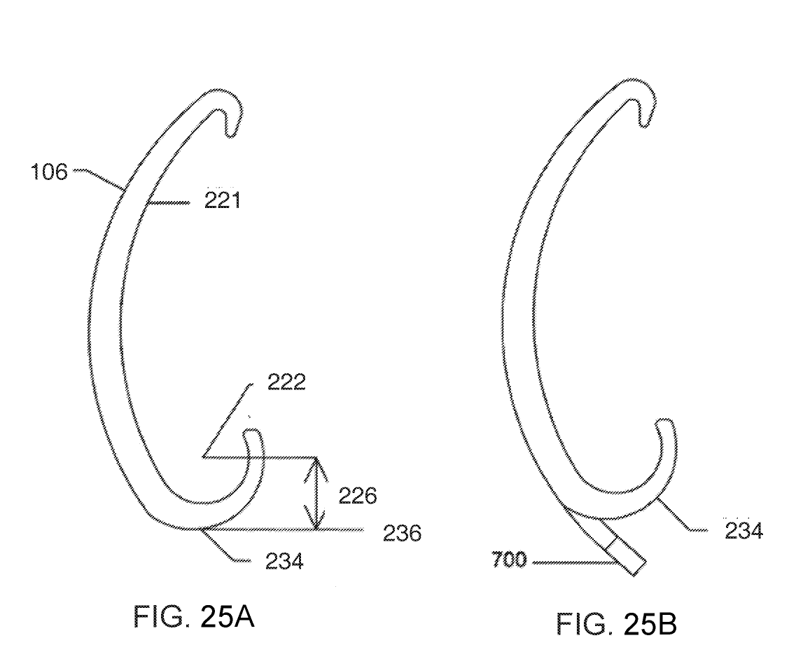

FIG. 25A is an illustration of a handle with open loop 234. Radius 222 is sized from 0.25 inches to 2.00 inches and is located at center point 226 which is 0.25 inches to 2.00 inches from baseline 236. The end of the open loop described can be contiguous and end at or before 230 of inner arc 221 of handle 300 with or without joining inner arc 221.

FIG. 25B is an illustration of a handle with suction attachment 700 at proximal end of open loop 234. The suction attachment 400 is sized to accept standard suction tubing (not shown) with inner diameters ranging from 0.0625 inches to 0.50 inches.

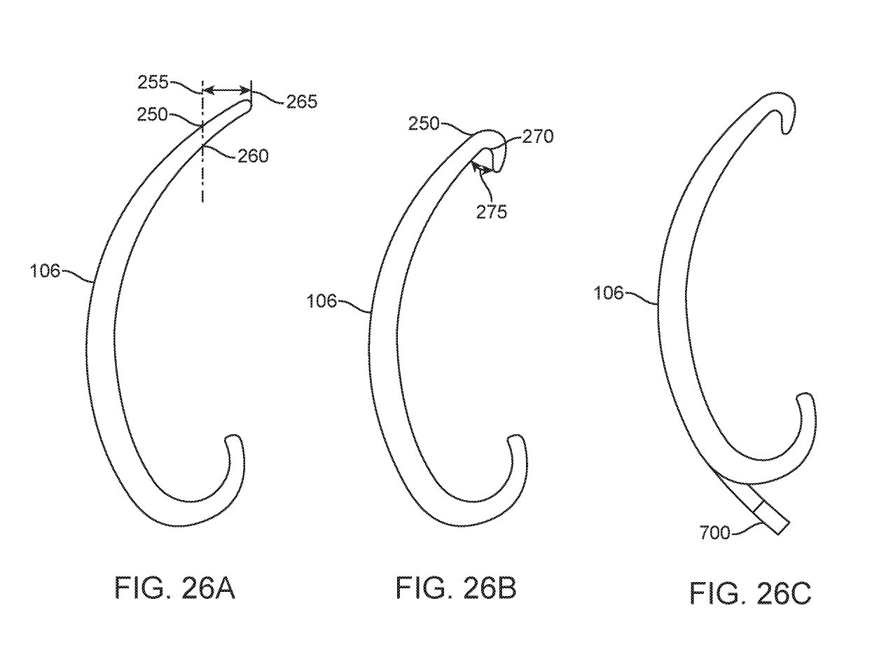

FIG. 26A is an illustration of handle 106 with formable distal tip 250 allowing for adjustment of purchase angle. The distal tip 250 is compliant and can be bent by hand at 260 on axis 255. It will take a set due to a looped wire backbone (not shown). The distance of the bended section can be adjusted from 0.125 inches to 0.75 inches long. The length of the formed tip 250 is defined by the length 230.

FIG. 26B is an illustration of handle 106 with formable distal tip 250. Angle 275 can be from 10 to 90 degrees and is defined by radius 270.

FIG. 26C is an illustration of handle 106 with suction attachment 700 sized to accept standard suction tubing (not shown) with inner diameters ranging from 0.0625 inches to 0.50 inches.

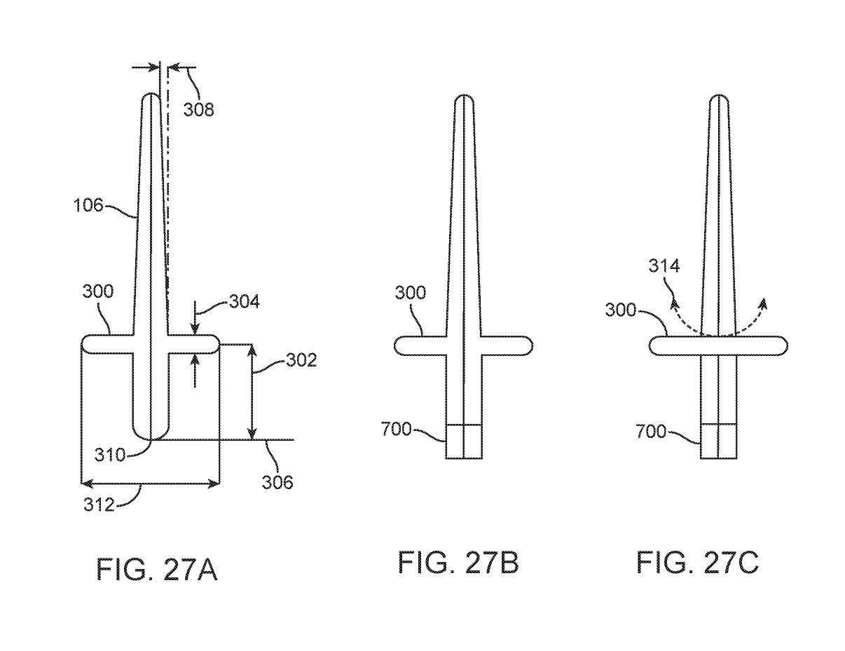

FIG. 27A is an illustration of end view of “T” handle 300. The height of the “T” is defined by the distance 302 from baseline 306 which can range from 0.50 inches to 10.0 inches in length. The diameter of each arm 304 can be from 0.125 inches to 0.50 inches. The shape of each arm 304 can be round, oval, square and hexagonal (not shown). The width 312 can be centered to the body or offset and can range from 1.00 inches to 4.00 inches in length. Additionally there is a radius 310 provided at the proximal end of the handle. Draft angle 308 is tapered distal to proximal with the distal end being of a smaller shape than the proximal end of the handle 106.

FIG. 27B is an illustration of the end view of the “T” handle 300 with suction attachment 700 sized to accept standard suction tubing (not shown) with inner diameters ranging from 0.0625 inches to 0.50 inches.

FIG. 27C is an illustration of the end view of the “T” handle 300 where the “T” 300 can rotate 360 degrees in either direction 314. The suction attachment 700 is sized to accept standard suction tubing (not shown) with inner diameters ranging from 0.0625 inches to 0.50 inches.

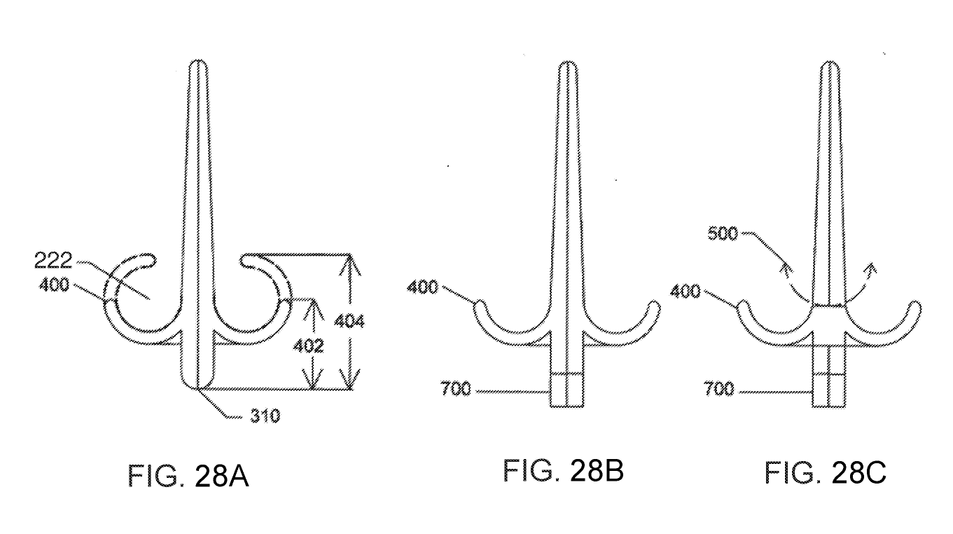

FIG. 28A is an illustration of the end view of a double open loop 400 configuration. The open radii 222 of open loop 400 can begin at 15 degree and continue through to 359 degrees. Open loop 400 is shown at 45 degrees 402. The open loop 400 is also shown (dotted line) at the 180 degree position 404. Additionally there is a radius 310 provided at the proximal end of the handle. These loops are intended to be used by the index and middle fingers predominantly but do not exclude the thumb and or index finger use.

FIG. 28B is an illustration of the end view of open loop handle 400 with the suction attachment 700 sized to accept standard suction tubing (not shown) with inner diameters ranging from 0.0625 inches to 0.50 inches.

FIG. 28C is an illustration of the end view of open loop handle 400 which is separately disposed on handle 106 and can rotate 500 separately. It is also provided with a suction tube attachment 700 which is proximal to the rotating handle 500 and is of a length so as to accept a standard suction tube (not shown) with inner diameters ranging from 0.0625 inches to 0.50 inches.

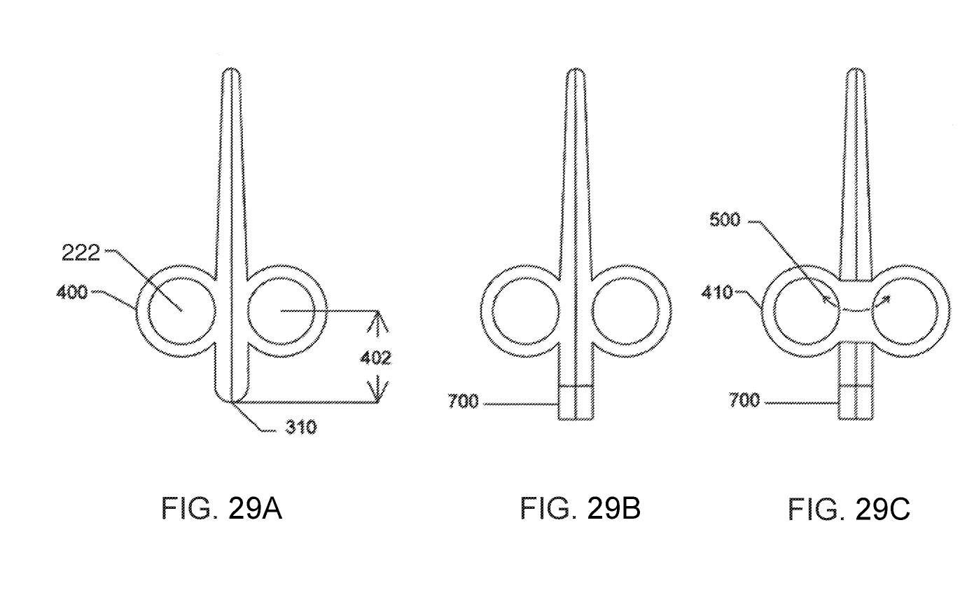

FIG. 29A is an illustration of a front view of handle 106 with double closed finger looped configuration 400. The radius 222 is between 0.25 inches and 2.00 inches. Its height from the proximal end of the handle 106 is defined by distance 402. 402 correlates directly with radius 222, if radius 222 is large then distance 402 must be in turn longer so as to accommodate larger finger loops. These loops are intended to be used by the index and middle fingers predominantly but do not exclude the thumb and or index finger use.

FIG. 29B is an illustration of the end view of open loop handle 400 with the suction attachment 700 sized to accept standard suction tubing (not shown) with inner diameters ranging from 0.0625 inches to 0.50 inches.

FIG. 29C is an illustration of the end view of closed loop handle 410 which is separately disposed on handle 106 and can rotate 500 separately. It is also provided with a suction tube attachment 700 which is proximal to the rotating handle 500 and is of a length so as to accept a standard suction tube (not shown) with inner diameters ranging from 0.0625 inches to 0.50 inches.

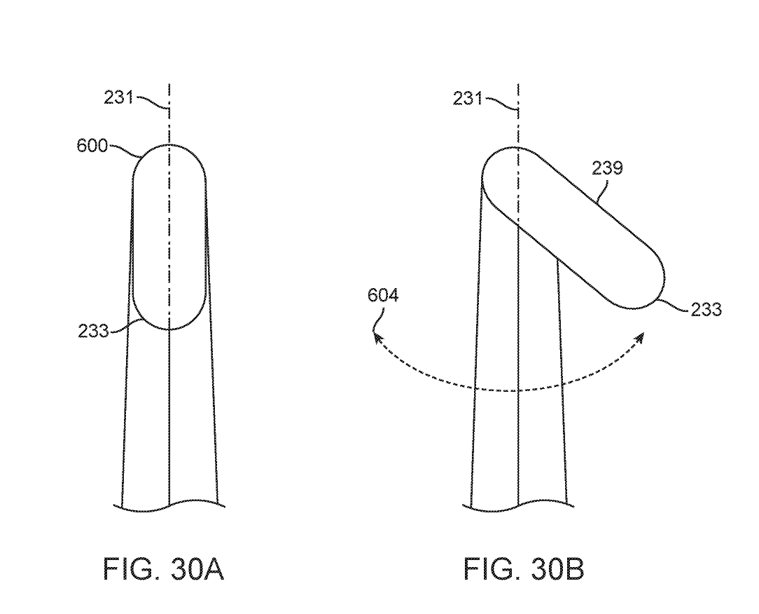

FIG. 30A is an enlarged view of the distal tip of handle 106 illustrating the front view of the hook component 223 in its normal or longitudinal position on longitudinal axis 231.

FIG. 30B is an enlarged view of the front view of the hook 223 in its swung or angled position 239 off of longitudinal axis 231. The hook 223 can be of various shapes and sizes—for example the hook can be primarily square, oval or triangular in nature or combinations of all or some.

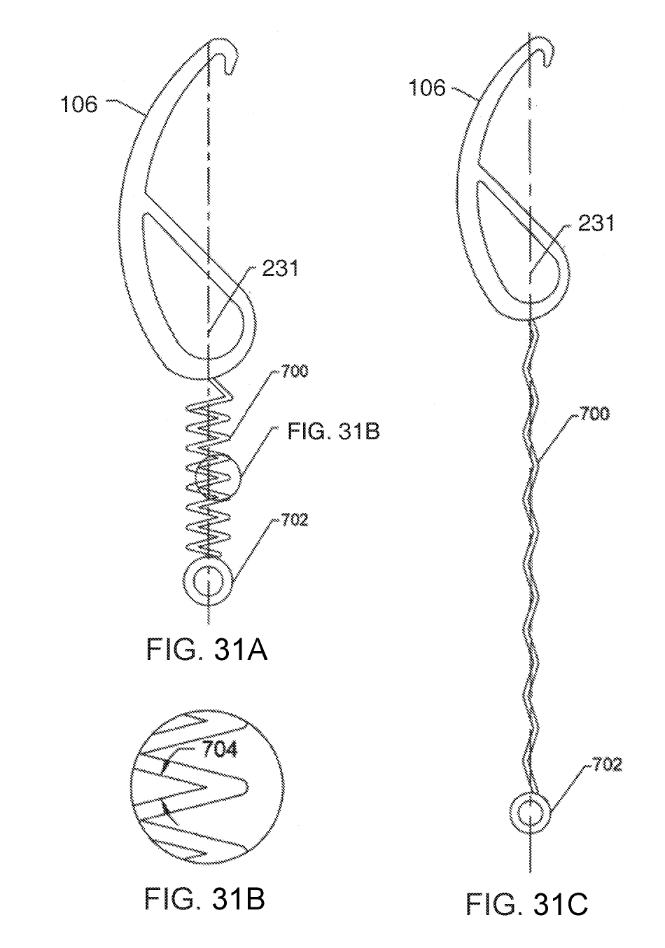

FIG. 31A is an illustration of a handle 106 with a secondary zig zag tether 700. It is provided at the proximal end of handle 106 and composed of an elastic material but not limited to silicone. It is independently assembled or it can be made as a continuation of a silicon or other elastic based handle 106. At the proximal most aspect of the zig zag element 700 is a finger loop 702 which can be made as a continuation of the zig zag element 700 or independently assembled to.

FIG. 31B is an enlarged view of 700 zig zag configuration of the elbow angle 704. The angle of each elbow can range from 5 to 30 degrees prior to expansion for instance and as much as 45 to 180 degrees as an example after traction on finger loop 702.

FIG. 31C is an illustration of the handle 106 under traction using finger loop 702 elongating the zig zag element 700.

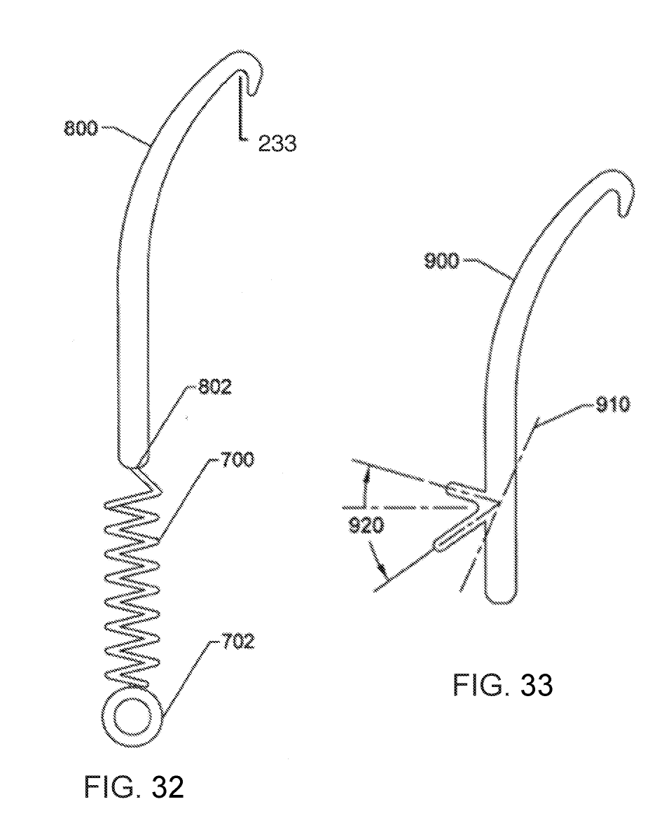

FIG. 32 is an illustration of a handle 800 provided with an elastic zig zag element 700 assembled to its most proximal end 802. Additionally, the handle 800 and zig zag element 700 could be made as a single unit whereby the durometer of the handle 800 would be of sufficient hardness such that hook 233 maintains its shape under traction while the zig zag element 700 is able to elongate using the finger loop 702.

FIG. 33 is an illustration of a handle 900 which is provided with “V” grip 920. “V” grip 920 is off axis from 910 and can range from 180 degrees of axis 910 to a minimum of 30 degrees 920 as an example.

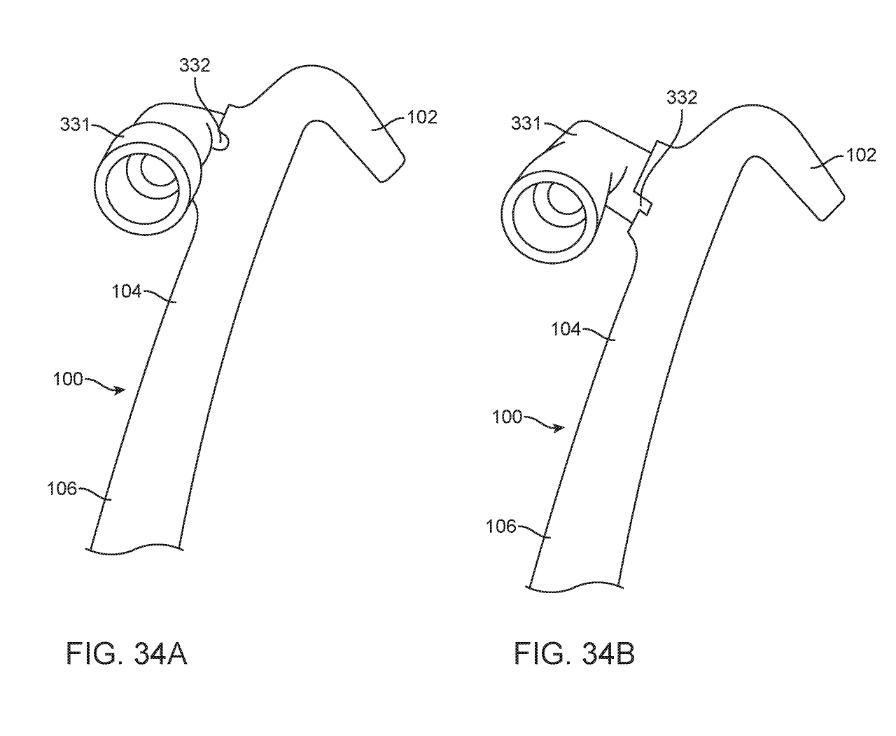

Referring again to FIGS. 17A-17B and 18, a device as disclosed herein may comprise a suction tube exiting from either the right or left side, or both sides, of the distal aspect of the handle of the device proximal to the tooth engaging portion. In some cases, such a configuration may be somewhat inconvenient during use as the dental practitioner may have to remove the device, remove the suction apparatus 200, remove the small caps from the nipple, place the suction apparatus 200 on the contralateral nipple, and place the small caps on the original nipple. To make changing from the right or left side to the other side easier, another embodiment of the device 100 shown in FIGS. 34A and 34B may comprise a swivel mechanism 331 that may be substituted for the elbow fitting in FIG. 18. FIG. 34A shows a shorter swivel mechanism 331 and FIG. 34B shows a taller swivel mechanism 331. The swivel mechanism may allow the elbow connection to rotate 360 degrees, but this may cause the suction apparatus to move about in the mouth and not maintain a selected position. In fact, most swivel configurations would not be stable and would require a détente or certain means 332 or mechanism to stabilize the suction apparatus once it is placed within the mouth. Other means and mechanisms to prevent the movement of the suction apparatus (not shown) once it is placed within the mouth and can be moved by the tongue, lips, dental instruments or even the dental practitioner are anticipated and included herein as potential embodiments. In a preferred embodiment, the rotation may be limited to 180 degrees or less and more preferably 130-160 degrees with the center of this rotational arc being the longitudinal axis of the handle 106 of the device and zero and 360 degrees being neck 104 and tooth engaging member 102. The suction connector is 180 degrees in this example. Hence, in a more preferred embodiment in which the swivel rotates 150 degrees, the swivel may rotate from 105 degrees to 265 degrees of the clock like 360 degree arc. To limit the rotation, a stop mechanism of one of various types on the outer surface of the neck 104 of the device may be employed. Alternatively, a stop mechanism within the swivel mechanism may be employed. By limiting the travel to 150 degrees in this example, the suction apparatus (not shown) is prevented from being displaced while in the mouth as it is prevented from rotating into the upward areas of the arc by the stops on the swivel. In this configuration, the upward pressures exerted by the lower lips tend to force the suction apparatus upward to a point where the stop prevents the suction apparatus from further upward movement. This upward pressure of the lips on the suction apparatus may pin the suction apparatus against the stop and stabilize the suction apparatus, preventing it from moving about when it is placed inside the mouth. The swivel configuration with the stop mechanism allows for the dental practitioner to change the suction apparatus from the right or left side by rotating it from approximately 8 o'clock to approximately 4 o'clock easily while preserving the stability of the device when it is placed at a precise location within the patient's mouth.

While most of the illustrations and the discussion reference a suction apparatus that is directed to only one side, bilateral suction apparatuses may be employed and will be employed in many instances. Other embodiments may utilize a suction that originates from the end of the tooth engaging portion 102 which may be modified to accept a suction tubing 130 as illustrated in FIGS. 34A and 34B.

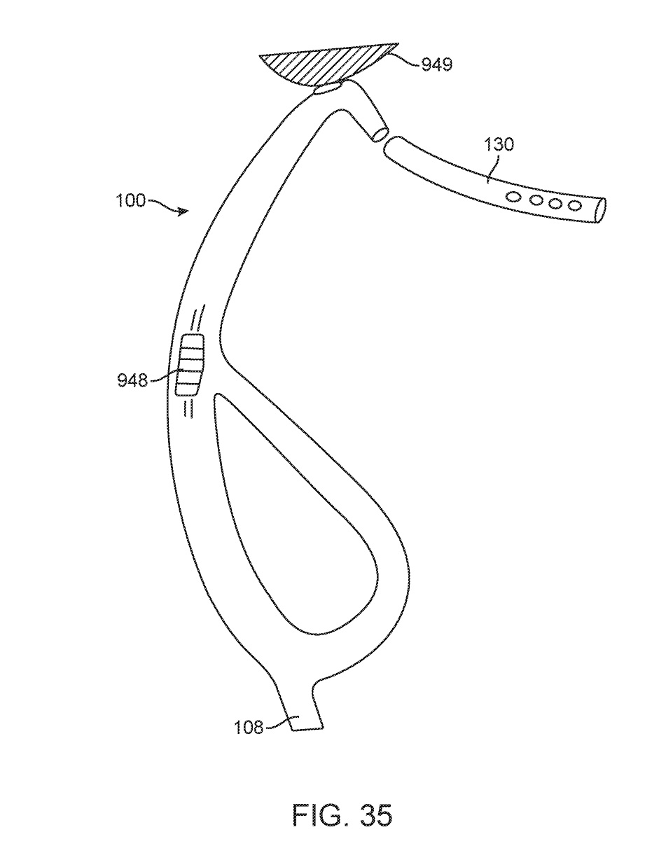

Frequently, with ultrasonic scaling and the use of copious amounts of water in the process, there is a large amount of water that collects in the posterior mouth and the foregoing embodiments coupled with suction may be very advantageous in removing this water. As well, there frequently is a large amount of splatter of water as it is sprayed onto the teeth. The splatter of droplets and mist is problematic as it escapes the oral cavity and wets the patient and hygienist as well as the surrounding environment. Another embodiment of the device previously described which distracts the jaw downward while providing suction may solve both problems, collection of water in the mouth and the spray and splatter of ricocheting water off the teeth, within the same device and it is illustrated in FIG. 35. Co-pending U.S. patent application Ser. No. 14/504,518, which is incorporated herein by reference, demonstrates a fluid suction tube extending into the posterior aspect of the mouth. A cup-like mist or splatter collector may be added to the devices shown and described with respect to FIGS. 10 and 13 of that application, along with a more or less straight perforated suction tube 130 with a controlling mechanism that will allow the patient to control the suction from either the suction tube or the mist collector. In FIG. 35, an improved embodiment may comprise a traction device 100 with suction tubing means 130 with distal perforations, a suction connector 108 on the opposite end, and a mist collector 949, which may be shaped like a shallow cup. The suction tubing 130 means may measure 2-8 mm in length, but optimally 6 mm, comprise an outer diameter of 1/16 inch-½ inch but optimally around ⅜ inch, and have a durometer of 40-60, but optimally around 60. The holes may be from one to 20, but optimally around 8 and they may be oriented in opposing pairs. The holes optimally have a diameter of 2.5 mm, but may range from 1 mm to 4 mm. The embodiment may preferentially be connected to the HVE suction commonly present in dental offices, although it may be connected to standard low volume suction.

While a free connection to both the fluid collector tubing 130 and the mist collector 949 may be feasible, a means may be present for directing the suction to one or the other to enhance the suction or vacuum. A slidable mechanism 948 or some other mechanism may direct the suction to either the suction tubing 130 or the funnel shaped mist collector 949. The slidable mechanism 948 may be activated with the patient's thumb or other digits. The slidable mechanism 948 may default suction to the mist collector 949 by a spring or other mechanism (not shown) when not engaged by the patient providing suction to the mist collector 949 until fluid evacuation is needed via the suction tubing 130. Hence, when the patient needs to evacuate fluid from the mouth, they may do so by a simple movement of the slidable mechanism 948 to direct suction toward the suction tubing means 130 and away from the mist collector 949. Upon removing fluid from the mouth, the patient may release the slidable mechanism 948 and the suction may then be directed to the mist collector 949. The slidable mechanism 948 may be located at any point along the shaft of the traction device 100 to be more ergonomic. Alternatively, when mist collection is not as important when cleaning the posterior teeth, the patient may utilize the fluid collector tubing 130 more than the mist collector 949.

The mist collector 949 is typically cup shaped to enhance collection of the mist but may take any one of other shapes. It may tilt in one or more planes with manual pressure or electronically so to be directed to the area of maximum spray, mist or splatter. Hence, this embodiment solves a lot of problems associated with ultrasonic scaling dental hygiene procedures by providing downward traction on the mandible and relaxing the muscles creating a comfortable atmosphere for the patient, a wider opening for the dental practitioner, fluid evacuation from the posterior mouth, spray and splatter collection preventing unwanted moisture outside the mouth, as well as involving the patient in the process which is psychologically important. Both the patient and the hygienist benefit.

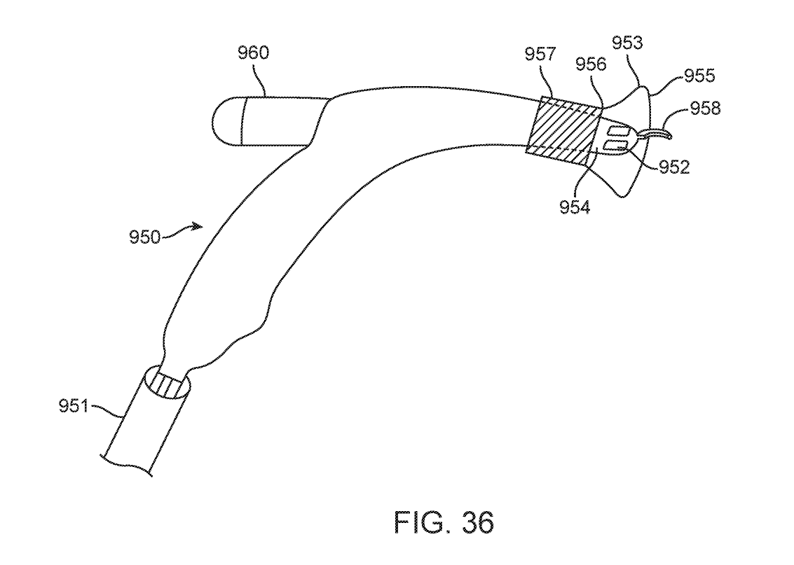

FIG. 36 illustrates a modification of an ultrasonic scaler hand piece 950 which in currently available scalers comprises at least one of a pressurized air and pressurized water supply. There also may be the option of adding additional materials to clean and polish the teeth to the fluid to a container 960 attached to the hand piece 950. The presence of both pressurized air and water exacerbates the splatter/spray phenomenon when cleaning the teeth. Even pressurized water alone causes significant spray or splatter. Hence, the dental hygienist must use separate devices to attempt to control the fluid which collects in the posterior oral cavity and to attempt to control the spray/splatter, while holding the ultrasonic scaler hand piece to clean the teeth. Since the hygienist only has two hands and needs three devices, this is frequently problematic, especially since the current fluid evacuator devices do not adequately remove the fluid and the suction apparatus used to control the spray/splatter only functions marginally. There is a needed improvement in the design of the current scaler hand pieces which are very efficient at cleaning but cause unwanted problems of fluid and spray/splatter control.

Some current ultrasonic scalers are connected to pressurized air or pressurized fluid or both via a tubing 951 which may be attached to the hand piece 950. The air and fluid may exit from the same or adjacent tubular openings 958 near the distal aspect of the hand piece 950. The improved embodiment of the current invention also may comprise a separate suction channel (not shown) in addition to either pressurized water or air or both. Apertures or holes 952 in the end of the hand piece 950 will suck the spray/mist caused by the pressurized air and water ricocheting off of the teeth into the suction channel within the hand piece (not shown). The suction channel within the hand piece is connected to a suction hose at the base of the hand piece and may be incorporated into the same hose 951 which contains the pressurized air and water channels.

To enhance the collection of more spray/mist, a funnel apparatus 953 which may be stationary, expandable and collapsible or directional may be attached to the distal aspect 954 of the hand piece. The funnel apparatus 953 may be only funnel like with a larger circumference at the mouth or opening 955 of the apparatus and a smaller circumference at the neck 956 or the point at which the apparatus is connected to the suction channel. Also, it may be a partial funnel like configuration with part of the funnel removed for visibility or other reasons (not shown). For example a half funnel or some other fraction of a funnel shaped apparatus may be utilized and positioned to gather the most reflected spray or splatter. The fractional funnel may also be rotatable around the end of the hand piece to be positioned for optimal suction, optimal visibility of the teeth being cleaned, or optimal access with other instruments or because of anatomy. One embodiment may comprise a nitinol mesh braid with an elastomeric coating, although there are many other fabrics and materials which may be employed. The funnel may be collapsible by one of several means. One means is a tubular member 957 which slides over the funnel 953 thereby collapsing the funnel 953 for improved access to posterior teeth or visibility or both. If the tubular member 957 covers the funnel 953, it will be collapsed in a tubular configuration. Retracting the tubular member 957 will allow the funnel 953 to expand into the useful funnel shape as illustrated in FIG. 36. An attachment (not shown) to the tubular member 957 may facilitate the movement of it to collapse and expand the funnel so that it may be partially expanded in a number of positions or completely expanded or completely collapsed. The funnel 953 may be from ¼ inch to 4 inches long, have a diameter at the mouth 955 of ½ inch to 4 inches. Alternatively the housing of the hand piece 950 may actually serve as the tubular member 957 to constrain the funnel, and expansion and collapsing of the funnel may be controlled by translating the funnel forward or backward. The funnel may be positioned closer or further away from the distal tip of the hand piece 950 than is illustrated in FIG. 36. An embodiment may include one or more of the features listed herein in different combinations.

There may be a means (not shown) of directing pressurized air, which is already present in the hand piece 950, into the funnel towards the neck 956 of the funnel 953 to enhance the direction of the water droplets or spray into the funnel 953 containing the suction so that the mist and spray is more efficiently removed via a Bernoulli or Venturi effect. The pressurized air directed toward the neck 956 of the funnel 953 will facilitate the flow of the mist/spray into the suction channel within the hand piece, obviating the current situation which tends to saturate the patient and dental hygienist with fluid.

Additionally, a surfactant which diminishes the surface tension of water, and hence creates smaller droplets, may be added to the air or water that is sprayed onto the teeth. This will serve to create lighter weight and smaller droplets that are more easily aspirated or sucked from the oral cavity as the lighter weight will prevent the smaller droplets from travelling as far. The combination of the funnel to collect the spray, the surfactant to cause the spray to be collected easier, and the suction to remove the spray will be more effective than any one method alone. Furthermore, by utilizing the jaw retraction device previously described in FIG. 35 with the device shown and described in reference to FIG. 36, the fluid which collects in the posterior oral cavity and the reflected spray/splatter may both be controlled much better than is done currently.

The improvements disclosed herein may be implemented with the devices described herein or with other suction devices that are commercially available including the Isolite, IsoDry, IsoVac, Mr. Thirsty, eBite, Miracle Suction, standard dental suction devices and the like. The subsequent improvements may be implemented with the embodiments of the current invention described herein or with other commercially available devices. Many of these latter devices combine suction with a bite prop that fits between the posterior teeth and a means of displacing the tongue and/or cheek away from the area to be treated. While somewhat efficient at removing fluid, they are not directed toward reducing the deflected water and resultant spray or splatter. Improving these devices and others by placing a means to collect the spray, which may be similar to the means described herein, on these devices may improve their function. The preceding discussion is directed toward improvements of a specific device, but they also may be applied to the class of devices that serve to remove fluid from the mouth. The means to improve suction of spray on these devices may consist of a shallow somewhat cupped shaped device that serves as a mist collector and may be connected to the suction channel within the device. As with the description herein of FIG. 35, there also may be a manual or electronic mechanism which regulates the amount of suction directed to either the existing intraoral suction apparatus or to the mist collector (not shown). If the mechanism is electronic, it may comprise a sensor that directs suction preferentially to the mist collector when there is little or no fluid flowing through the standard intraoral fluid collection apparatus, but when significant fluid is present through the standard intraoral fluid collector system, more suction is directed toward it. Alternatively, the suction directed toward the mist collector may be synchronized with the air and water spray of the ultrasonic scaler hand piece. In other words, the mist collector suction may be operable only when the hand piece is activated for cleaning purposes, including set times before and after the hand piece activation, and essentially only when there is spray to collect. This time may vary from one tenth of a second to ten seconds, although the suction preferentially would begin ½ second before hand piece activation and three seconds after hand piece activation. The mechanism may be all or none or partial, i.e., directing all of the suction toward the intraoral apparatus or the mist collector, or directing only a portion of the suction toward the intraoral apparatus and another portion of the suction to the mist collector and it may be variable between the two suction apparatuses.

As well, the improvements may comprise adding a positive air pressure means to these isolation/suction devices to achieve one or more of facilitating the suction by creating a Bernoulli/Venturi effect and enhancing suction, or to dry the air to enhance evaporation of the spray. The means to provide positive air pressure may consist of a separate tube which may be added to or incorporated within the suction tubing of this class of suction devices and controlled by means known in the industry. Specifically fabricated connectors to connect both the suction and positive air pressure channels to the respective suction/vacuum and pressure sources within a dental office may be utilized. The air may preferentially be directed toward the neck or bottom of the cup like or funnel like mist collector to direct moisture laden air into the suction channel connected to the mist collector. Channels that direct the positive air pressure to points near the free end of the mist collector may be employed so that air may be directed toward the neck of the funnel like shape. As well, the positive air pressure may comprise of a means to subject the air to a desiccant or to warm the air or both which may be incorporated into the improved device or may be a separate attached device.

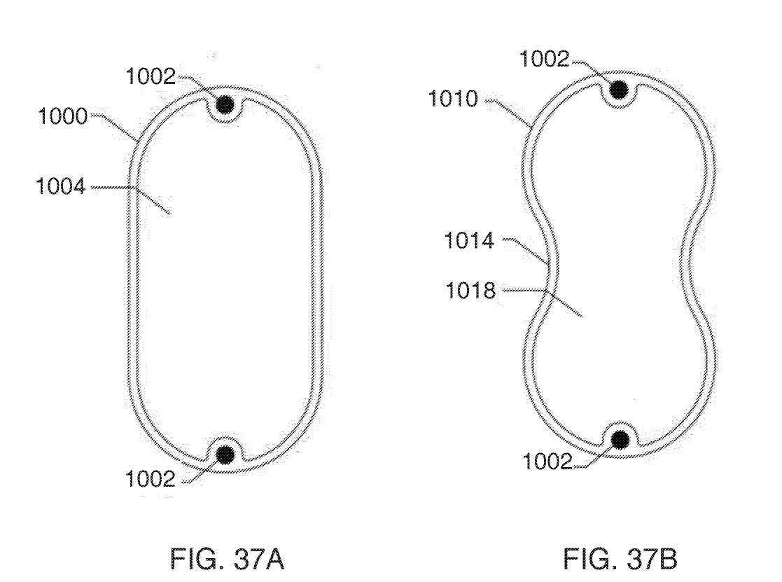

FIG. 37A is a transverse cross-section view of an exemplary embodiment of a suction tube for insertion into the mouth, suitable for incorporation with any mouth opening device as described herein. The suction tube 1000 may comprise an oval-shaped cross-section, so as to fit more comfortably between the gum line inside the mouth. The suction tube may further comprise one or more support wires 1002 coupled to the tube. The support wires are malleable and can be bent numerous times so as to allow for adjustments as needed during the oral procedure. Any number of support wires may be provided with the suction tube, and the support wires may be coupled to the tube using any appropriate method. For example, as shown in FIG. 37A, the suction tube may comprise two support wires capture-molded within the tubing material, at opposing ends along the major longitudinal axis 1006 of the oval-shaped cross-section (e.g., at 12 o'clock position and 6 o'clock position as shown). The capture-molding of the support wires within the tubing material can allow for a clear aspiration lumen 1004.

FIG. 37B is a transverse cross-section view of an exemplary embodiment of a suction tube for insertion into the mouth, suitable for incorporation with any mouth opening device as described herein. The suction tube 1010 may comprise a generally oval-shaped cross-section with a waist section 1014, the waist section having a width 1015 that is smaller than the maximum width 1011 of the cross-section. The suction tube may further comprise one or more support wires 1002 substantially similar to the support wires described in reference to the embodiment of FIG. 37A. For example, the support wires may be provided at opposite ends along the major longitudinal axis 1016 of the substantially oval-shaped cross-section, and the support wires may be capture-molded within the tubing material so as to not impede flow through aspiration lumen 1018. The support wires may be malleable and capable of being bent numerous times to allow for adjustments as necessary.

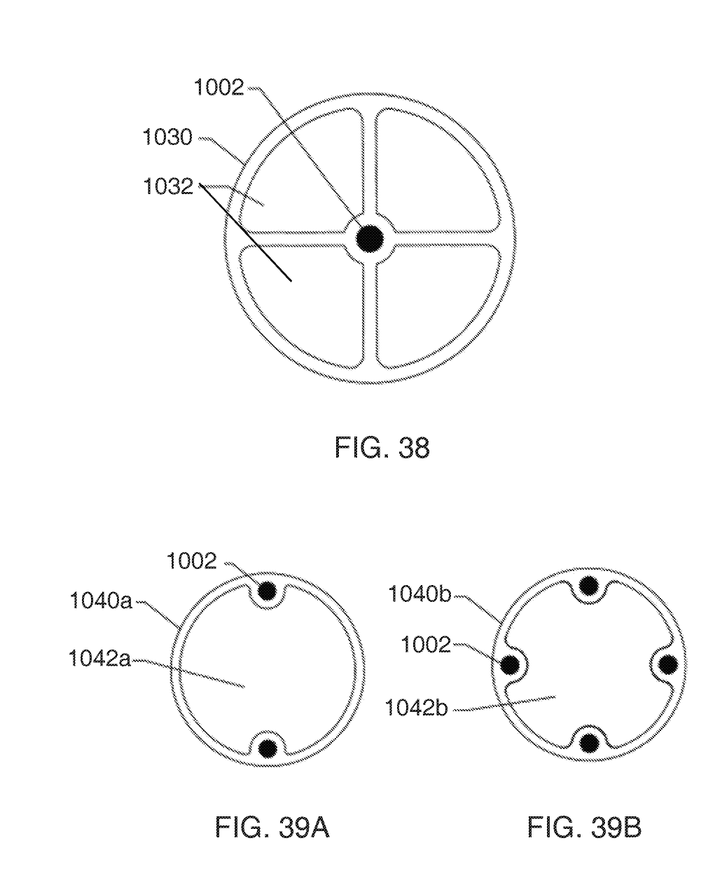

FIG. 38 is a transverse cross-section view of an exemplary embodiment of a multi-lumen suction tube, suitable for incorporation with any mouth opening device as described herein. The suction tube 1030 may comprise one or more support wires 1002 as described herein, extending axially throughout the length of the suction tube. The suction tube may comprise a plurality of separate lumens 1032 extending axially through the length of the suction tube. The one or more support wires may be capture-molded within the tubing material, such that each of the plurality of lumens remains unobstructed by the support wires. While the embodiment of FIG. 38 shows the suction tube 1030 having a substantially circular cross-section with a single support wire disposed at the center of the circular cross-section, suction tube may have any cross-sectional shape, and may comprise any number of support wires disposed at any appropriate location(s) within the tube. Similarly, while the embodiment of FIG. 38 shows four lumens 1032 of equal shape and size distributed radially symmetrically about the longitudinal axis of the suction tube, the suction tube may comprise any number of lumens having any shape, size, or distribution within the suction tube.

FIG. 39A is a transverse cross-section view of another exemplary embodiment of a suction tube, suitable for incorporation with any mouth opening device as described herein. The suction tube 1040a may have a substantially circular cross-sectional shape. The suction tube may further comprise one or more support wires 1002 as described herein, capable of being bent numerous times to allow for adjustment. For example, the suction tube may comprise two support wires 1002, disposed at opposite ends across the diameter of the suction tube. The support wires may be capture-molded within the tubing material to allow for a clear suction tube lumen 1042a.

FIG. 39B is a transverse cross-section view of another exemplary embodiment of a suction tube, suitable for incorporation with any mouth opening device as described herein. The suction tube 1040b is similar in many aspects to suction tube 1040a shown in FIG. 39A, except the suction tube 1040b comprises four support wires 1002. The support wires are capture-molded in the tubing material so as to allow for a clear flow through the aspiration lumen 1042b. In this exemplary embodiment four support wires are distributed in a radially symmetric manner about the circumference of the tube, such that each support wire is radially offset by 90° with respect to an adjacent support wire. Such a configuration allows for bending of the suction tube in all planes. The configuration of FIG. 39B is shown by way of example only, and the suction tube may incorporate a smaller or greater number of support wires distributed in any manner within the suction tube.

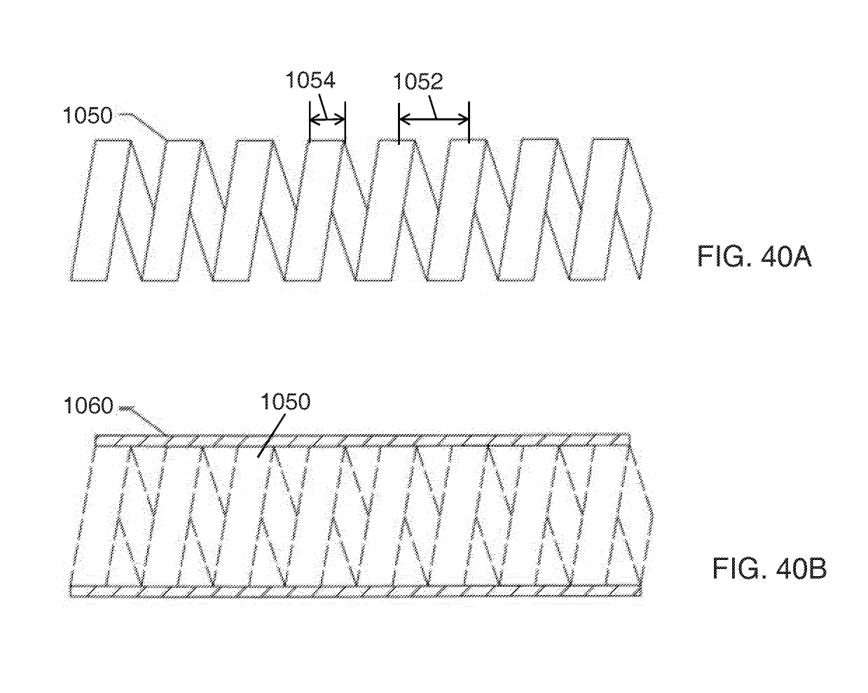

FIG. 40A shows an exemplary embodiment of a helical ribbon of a suction tube, suitable for incorporation with any mouth opening device as described herein. The helical ribbon 1050 may be disposed about the periphery of a suction tube, for example capture-molded within the tubing material, coupled to the internal surface of the tube facing the lumen, or coupled to an external surface of the tube. The helical ribbon may comprise a malleable wire configured to have a spring-type shape, for example. The helical ribbon may have a tight or elongated pitch 1052 as desired or necessary. For example, the helical ribbon may be configured to have pitch that is long enough to prevent stacking of the winds when the ribbon is bent at an acute angle. A relatively elongated pitch may have the advantage of allowing for a greater sweep of the suction tube or a more acute bend angle. The width 1054 of the helical ribbon may also be adjusted to control the range of motion of the suction tube. A relatively narrower ribbon may allow for a greater bending range due to reduced stacking on the winds during bending, while a relatively wider ribbon may result in quicker stacking of the winds during bending and thereby achieve less acute bending angles.

FIG. 40B shows an exemplary embodiment of suction tube comprising a helical ribbon, suitable for incorporation with any mouth opening device as described herein. The suction tube 1060 comprises an integrated helical ribbon 1050, which may be similar in many aspects to the helical ribbon 1050 shown in FIG. 40A. In this embodiment, the helical ribbon is capture-molded within the tubing material such that the aspiration lumen remains clear. The helical ribbon may comprise a malleable material that is capable of bending numerous times to allow for adjustment as needed during the procedure.

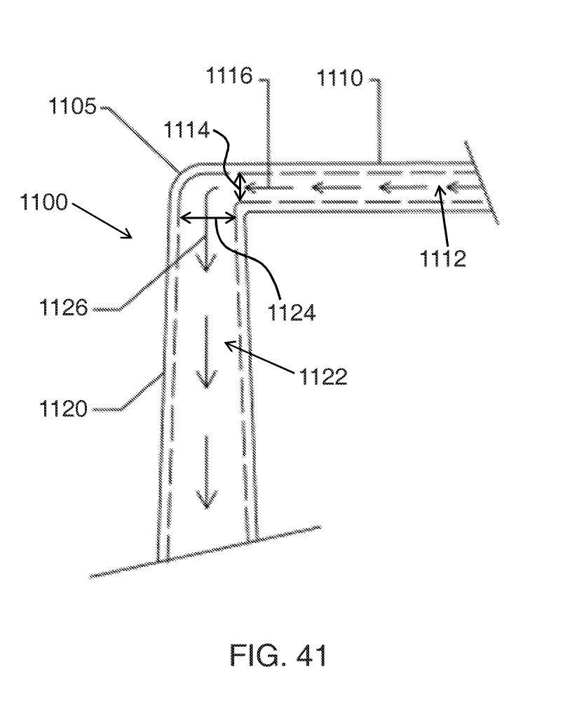

FIG. 41 is a schematic illustration of an exemplary embodiment of a suction tube suitable for incorporation with any mouth opening device as described herein. The suction tube 1100 comprises a mouth suction tube 1110 for insertion into the mouth and a main suction tube 1120. The mouth suction tube and the main suction tube may be separate components coupled together at the juncture 1105, or they may be different portions of a single, integrated suction tube, wherein the mouth suction tube portion transitions into the main suction tube portion at juncture 1105. The mouth suction tube may have a first aspiration lumen 1112 with a first internal diameter 1114, and the main suction tube 1110 may have a second aspiration lumen 1122 with a second internal diameter 1124, wherein the first and second aspiration lumens are fluidly coupled at the juncture 1105. Each of the first and second internal diameters of the aspiration lumens may vary over the length of the suction tubes, as shown in FIG. 41 for the second aspiration lumen of the main suction tube, whose internal diameter increases along the length of the main suction tube in the direction of flow of the aspirate. In preferred embodiments, at the juncture 1105, the internal diameter 1114 of the first aspiration lumen 1112 is smaller than the internal diameter 1124 of the second aspiration lumen 1122, as shown in FIG. 41. The difference in the internal diameters of the lumens can lead to differences in the aspiration rates through the two aspiration lumens, with the first aspiration rate 1116 through the first aspiration lumen 1112 greater than the second aspiration rate 1126 through the second aspiration lumen 1122. The correlation between the aspiration lumen diameters and the aspiration rates can be summarized by the following equation:

D1<D2, therefore V1>V2

wherein D1 is the internal diameter 1114 of the mouth suction tube lumen at the juncture 1105, D2 is the second aspiration lumen diameter 1124 of the main suction tube lumen at the juncture, V1 is the first aspiration rate 1116 through the mouth suction tube, and V2 is the second aspiration rate 1126 through the main suction tube.

All of the physical components of suction and the actions described herein, including but not limited to suction and positive air pressure, may be synchronized or activated with the scaler or other dental hand piece, may be activated by a foot pedal or the like, may be activated by manual activation by the patient or dental practitioner, or controlled by a like means.

The suction inlets in all of the examples herein may be directly connected to the suction channel which extends through the suction connector and is continuous with the suction channel of the suction valve. As well, any one feature may be combined with any other feature described herein, and the specific features mentioned should not be limited to a specific embodiment or species.

The devices described herein comprise different components and various configurations of these different components. The single or multiple configurations of the separate components may be combined with any single or multiple configuration of another component or components that may result in a device not explicitly described herein. By providing this flexibility in the structural configuration of various devices, the goals of providing the dental practitioner with greater exposure and convenience and the patient with greater comfort can be achieved.

### EXPERIMENTAL EXAMPLES

In using a device as described herein, the tooth piece may be placed over the lower front teeth by the dental practitioner or the patient, and the patient may secure the handheld apparatus. To maximize the effectiveness, the patient can place downward pressure on the device and the mandible while consciously urging the mandible upward. This may be a strain against the downward force. The patient may then relax and stretch the muscles with continued downward force on the device. The patient may repeat this exercise one or more times, and then relax the jaw muscles and pull downward tension more or less continuously for a period of time. This method has, in a small trial, demonstrated a greater degree of measurable opening of the mouth with little or no discomfort more than any other method. During interludes when the dental practitioner is not occupied within the mouth, the patient may briefly rest by closing the mouth. Then downward traction or tension may be exerted on the device and mandible again and maximum opening achieved.

In testing this device and method, patients that actively opened the mouth without any assistance from any device lost an average of 10-25% of the original opening distance after 30 minutes in the procedure. Patients tested using a mouth opening device and method described herein gained 10-30% of the original opening distance at the 30 minute period. The patients with the assist device and method were also more comfortable than patients without the assist device and method. Hence, the combination of the devices and methods described herein benefited the dentist by providing significantly more exposure and the patient by obviating the pain usually experienced with this procedure.

Although the above description is complete and accurate, it is not meant to be exhaustive or to limit the scope of the invention beyond what is set forth in the following claims. Various alterations, modifications, additions, and deletions may be made to any of the devices and methods, without departing from the scope of the invention.

1.-10. (canceled)

11. A device for helping maintain a mouth of a patient in an open position during a dental procedure, the device comprising:
a tooth engaging portion comprising at least one groove for accepting at least one lower tooth of the patient's lower jaw;
an extension portion that extends away from the tooth engaging portion;
a handle that extends down from the extension portion and ends in a caudal end; and
a suction tube coupled to at least one of the tooth engaging portion, extension portion, or handle,
wherein the suction tube has a suction inlet configured to be placed in the patient's mouth when the at least one groove accepts the at least one lower tooth to provide suction to the patient's mouth, and
wherein the handle comprises a suction connector configured to connect to a suction source.

12. The device of claim 11, wherein at least a portion of the suction tube comprises one or more malleable support wires to enable bending of the suction tube.

13. The device of claim 12, wherein the one or more support wires are capture molded within a material of the suction tube to keep a lumen of the suction tube clear.

14. The device of claim 11, wherein the suction tube comprises a plurality of lumens.

15. The device of claim 11, wherein at least a portion of the suction tube comprises a helical ribbon, the helical ribbon configured to enable bending of the suction tube.

16. The device of claim 11, wherein the suction tube is attached to the tooth engaging portion and is configured to be directed into one of the lingual sulcus or tonsillar fossa or adjacent to the tongue when the tooth engaging portion is accepting the at least one lower tooth.

17. The device of claim 11, wherein the suction inlet is coupled to the at least one of the tooth engaging portion, extension portion, or handle at a juncture, wherein the suction inlet comprises a first lumen having a first diameter at the juncture, and wherein the at least one of the tooth engaging portion, extension portion or handle coupled to the suction tube comprises a second lumen having a second diameter at the juncture, and wherein the second diameter is greater than the first diameter.

18. The device of claim 17, wherein the second lumen is configured to have an aspirate move therethrough at a second aspiration rate, and wherein the first lumen is configured to have the aspirate move therethrough at a first aspiration rate greater than the second aspiration rate.

19. The device of claim 11, further comprising
a tissue displacement member coupled to at least one of the tooth engaging portion, extension portion, or handle, wherein the tissue displacement member is configured to displace at least one of a cheek or tongue of the patient when the at least one groove of the tooth engaging portion is accepting the at least one lower tooth.

20. A device for helping maintain a mouth of a patient in an open position during a dental procedure, the device comprising:
a tooth engaging portion comprising at least one groove for accepting at least one lower tooth of the patient's lower jaw;
an extension portion that extends away from the tooth engaging portion; and
a handle that extends down from the extension portion and ends in a caudal end,
wherein the caudal end of the handle and the at least one groove of the tooth engaging portion lie along a longitudinal axis drawn through a center of the at least one groove.

21. The device of claim 20, wherein the device further comprises a suction tube that is attached to the tooth engaging portion and is directed into one of the lingual sulcus, the tonsillar fossa or adjacent to the tongue.

22. The device of claim 20, wherein a distance between the caudal end of the handle and a junction of said tooth engaging portion and said handle is between 4 and 10 inches.

23. The device of claim 22, wherein an angle at said junction is between 30 and 90 degrees.

24. The device of claim 21, wherein the tooth engaging portion comprises a fitting that is attached to said suction tube.

25. The device of claim 20, wherein said handle comprises a means of securing traction by the patient in the form of one of a loop, T shape, hook, or other means.

26. The device of claim 25, wherein said means is adjustable or moveable in relation to said handle.

27. The device of claim 20, wherein the device further comprises a suction tube that is attached to a distal aspect of the handle and extends to one or both lateral sides of the handle.

28. The device of claim 27, wherein an attachment means of the suction tube to said handle is rotatable from one lateral side of the handle to another lateral side of the handle.

29. The device of claim 28, wherein said rotatable attachment means comprises a stop to limit the rotation to a defined arc.

30. The device of claim 20, wherein the device further comprises a non-tubular suction apparatus disposed near a junction of said handle and said tooth engaging portion.

31.-32. (canceled)

33. A method of maintaining a mouth of a patient in an open position during a dental procedure, the method comprising:
placing a groove of a tooth engaging portion of a mouth distraction device over at least one lower tooth in a lower jaw of the patient's mouth;
pulling down on a handle of the mouth distraction device in a direction along a longitudinal axis of the at least one lower tooth to maintain the lower jaw in an open position relative to an upper jaw of the patient's mouth; and
applying suction in the patient's mouth, using a suction device attached to one or more of the tooth engaging portion or the handle,
wherein the groove of the tooth engaging portion of the mouth distraction device and the handle of the tooth distraction device lie along a common, distraction device longitudinal axis, and
wherein the distraction device longitudinal axis lies along the longitudinal axis of the at least one tooth during the pulling step.

34. The method of claim 33, wherein the suction device collects both fluid from a dependent portion of the mouth and mist or spray from a different portion of the mouth.
